(19) United States
(12) Patent Application Publication
(10) Pub. No.: US 2025/0121068 A1
(43) Pub. Date: Apr. 17, 2025

(54) BIFUNCTIONAL MOLECULES AS PROTEASOME STIMULATORS FOR IMPROVED TARGETED PROTEIN DEGRADATION, AND THEIR USES AS TARGETED BOOSTING DEGRADERS (TARBODS)

(71) Applicant: BOOSTER THERAPEUTICS GMBH, Berlin (DE)

(72) Inventors: Diogo FELECIANO, Berlin (DE); Jens ECKSTEIN, Cambridge, MA (US); Darci TRADER, West Lafayette, IN (US)

(21) Appl. No.: 18/689,611

(22) PCT Filed: Sep. 9, 2022
(86) PCT No.: PCT/EP2022/075119

§ 371 Date: Mar. 6, 2024

Related U.S. Application Data
(60) Provisional application No. 63/242,235, filed on Sep. 9, 2021.

(30) Foreign Application Priority Data
Sep. 15, 2021 (EP) 21196945.6

(51) Int. Cl. A61K 47/54; A61K 47/55; A61P 25/28

(52) U.S. Cl. CPC A61K 47/545; A61K 47/55; A61P 25/28

(57) ABSTRACT

The present invention relates to molecules, pharmaceutical compositions and methods of use for the treatment and/or prevention of a disease or condition caused by an insufficient degradation of proteins by the proteasome system. The present invention particularly relates to a bispecific binding molecule, wherein said compound is an effective stimulator of the 20S core particle (CP) of the proteasome.

The present invention relates to molecules, pharmaceutical compositions and methods of use for the treatment and/or prevention of a disease or condition caused by an insufficient degradation of proteins by the proteasome system. The present invention particularly relates to a bispecific binding molecule, wherein said compound is an effective stimulator of the 20S core particle (CP) of the proteasome.

### BACKGROUND OF THE INVENTION

Targeted protein degradation (TPD) offers a novel therapeutic alternative by inducing the depletion or reduction of a disease-causing protein via hijacking the endogenous protein degradation machineries. TPD has the potential to target the undruggable proteome that limits current drug discovery efforts, as only a binder is required to recruit the target protein for degradation rather than high affinity inhibitors.

The major protein degradation pathway in cells is the ubiquitin-proteasome system (UPS). This system involves a network of proteins to polyubiquitinate and degrade protein substrates. Proteins are tagged for degradation with a small protein called ubiquitin. The tagging reaction is catalyzed by enzymes called ubiquitin ligases. Once a protein is tagged with a single ubiquitin molecule, this is a signal to other ligases to attach additional ubiquitin molecules. The result is a polyubiquitin chain that is bound by the proteasome, allowing it to degrade the tagged protein.

The degradation process is performed by the 26S proteasome, which is comprised of a 19S regulatory particle (19S RP) and a 20S core particle (20S CP). The 20S CP is responsible for the hydrolysis activity, degrading proteins into shorter peptides, and is regulated by the 19S RP, which recognizes ubiquitinated substrates, removes ubiquitin, and coordinates the movement of the substrate into the catalytic core particle for degradation.

The 20S CP alone can accept and degrade proteins in a ubiquitin-independent system (UIPS). In this case, proteins are not ubiquitinated and must be disordered enough to enter the catalytic core without being denatured by the 19S RP. The UIPS has been shown to play an important role in the degradation of oxidatively damaged proteins during times of cellular stress.

Aging is a natural process accompanied by a progressive accumulation of damage in all constituent macromolecules (nucleic acids, lipids and proteins). Accumulation of damage in proteins leads to failure of proteostasis (or vice versa) due to increased levels of unfolded, misfolded or aggregated proteins and, in turn, to aging and/or age-related diseases.

The proteasome and the lysosome have been shown to dysfunction during aging and age-related diseases, Parkinson's, and Alzheimer's disease. Regarding the proteasome, it is well established that it can be activated either through genetic manipulation or through treatment with natural or chemical compounds that eventually result to extension of lifespan or deceleration of the progression of age-related diseases. Stimulation of the 20S CP has recently been shown to be promoted by small molecules, such as AM-404 (AM), an AM-404 derivative (TRC-1), ursolic acid (UA), and miconazole (MO), leading to a more rapid degradation of disordered proteins (see, for example, Coleman, Rachel A, et al.; Protein degradation profile reveals dynamic nature of 20S proteasome small molecule stimulation. 2021, 636-644, RSC Chem. Biol. 2, doi:10.1039/DOCB00191K). In biochemical proteasome activity assays, MO and AM404 displayed the greatest stimulatory activity, increasing 20S CP activity more than 400% (at 25 μM) over the basal level control.

Njomen and Tepe (in: Proteasome Activation as a New Therapeutic Approach To Target Proteotoxic Disorders. J Med Chem. 2019; 62(14):6469-6481. doi:10.1021/acs.jmedchem.9b00101) review current approaches, genetic manipulation, posttranslational modification, and small molecule proteasome agonists used to increase proteasome activity, challenges facing the field, and applications beyond aging and neurodegenerative diseases. Molecules that interact with either the 20S or both the 20S and 26S proteasome, function through two main mechanisms, namely: (1) Gate-openers: Molecules that promote substrate entry into the catalytic pocket through allosteric interactions that induce gate-opening in the alpha ring of the 20S proteasome. (2) Stimulators: Molecules that promote 20S and/or 26S-mediated degradation through allosteric interactions that enhance substrate binding and/or degradation in one or more catalytic sites. Among the first reported 20S agonists is the triterpenoid, betulinic acid, which was reported to specifically enhance the CT-L activity of the 20S proteasome. Unfortunately, chemical modifications to improve activity resulted in proteasome inhibitors with complicated structure activity relationships (SAR).

WO 2018/064589 prepared bifunctional compounds for targeted protein degradation, that function to recruit targeted proteins to a functional, mutant E3 ubiquitin ligase, for example a functional, mutant cereblon, resulting in the ubiquitination of the targeted protein and subsequent proteasomal degradation.

WO 2017/011371 relates to MDM2 binding compounds, including bifunctional compounds comprising the same, which find utility as modulators of targeted ubiquitination.

WO 2021/034627A1 relates to series compounds and methods of use for the treatment of a disease caused by abnormal regulation of the ubiquitin-proteasome system (UPS), and wherein said compound is an effective stimulator of the 20S core particle (CP) of the UPS.

Tomoshige S et al. (in: Discovery of Small Molecules that Induce the Degradation of Huntingtin. Angew Chem Int Ed Engl. 2017 Sep. 11; 56(38):11530-11533. doi: 10.1002/anie.201706529. Epub 2017 Aug. 9. PMID: 28703441) disclose two small hybrid molecules (1 and 2) by linking a ligand for ubiquitin ligase (cellular inhibitor of apoptosis protein 1; cIAP1) with probes for mHtt aggregates, anticipating that these compounds would recruit cIAP1 to mHtt and induce selective degradation by the ubiquitin-proteasome system.

In contrast to the above, the present molecules function independently of ubiquitination. Coleman R A, et al. (in: Protein degradation profile reveals dynamic nature of 20S proteasome small molecule stimulation. RSC Chem Biol. 2021 Jan. 5; 2(2):636-644. doi: 10.1039/dOcb00191k. PMID: 34458805; PMCID: PMC8341874) discuss that small molecules have been discovered to stimulate the 20S core particle (CP) of the proteasome to degrade proteins. They evaluate the effects of two stimulators on the whole cellular proteome in HEK-293T cells using label-free quantitative proteomic analysis for a broader understanding on their impact.

Other technologies that induce targeted protein degradation by small molecules have been developed recently. Chimeric small molecules such as Proteolysis Targeting Chimeras (PROTACs) and Specific and Non-genetic IAP-dependent Protein Erasers (SNIPERs), and E3 modulators such as thalidomides, hijack the cellular machinery for ubiquitylation, and the ubiquitylated proteins are subjected to proteasomal degradation. This has motivated drug development in industry and academia because “undruggable targets” can now be degraded by targeted protein degradation (Naito M, Ohoka N, Shibata N, Tsukumo Y. Targeted Protein Degradation by Chimeric Small Molecules, PROTACs and SNIPERs. Front Chem. 2019 Dec. 10; 7:849. doi: 10.3389/fchem.2019.00849. PMID: 31921772; PMCID: PMC6914816). PROTACs consist of a target protein ligand connected via a short linker to an E3 ligand, allowing PROTACs to function as bridging compounds that bring an E3 into proximity with specific cellular proteins. The juxtaposition of the E3 complex and target protein facilitates the processive transfer of ubiquitin from the E3 complex to the target protein, thereby tagging the protein for degradation via the proteasome.

WO 2020/041331A1 discloses such proteolysis targeting chimeric (protac) compound with E3 ubiquitin ligase binding activity and targeting alpha-synuclein protein for treating neurodegenerative diseases. Disclosed are bifunctional compounds, which contain on one end a Von Hippel-Lindau, cereblon, Inhibitors of Apotosis Proteins or mouse double-minute homolog 2 ligand which binds to the respective E3 ubiquitin ligase and on the other end a moiety which binds the target protein. The target protein is placed in proximity to the ubiquitin ligase to effect degradation (and inhibition) of target protein. Diseases or disorders to be treated are alpha-synucleinopathies or neurodegenerative diseases associated with alpha-synuclein accumulation and aggregation, such as e.g., Parkinson Disease, Alzheimer's Disease, dementia, dementia with Lewy bodies or multiple system atrophy, in particular Parkinson's Disease.

While empiric guidelines like the rule-of-five (C. A. Lipinski, et al., Adv. Drug Delivery Rev. 1997, 23, 3) for the optimization of traditional orally available small molecules inhibitors have been developed and refined over the past decades, optimizing the bioavailability for large molecules like PROTAC is still in its infancy. Optimizing a beyond rule-of-five compound with a molecular weight most likely between 700 and 1200 g mol⁻¹ for clinical applications is an enormous challenge as many principles established for small molecule inhibitors do not translate to larger molecules. With only very few design guidelines available PROTAC optimization is still no straight forward process.

The development of more effective and selective molecules mediating proteasome degradation of undesired proteins is needed if they are to become useful in the treatment and/or prevention of diseases where undesired proteins and protein accumulation plays a role. It is an object of the present invention to provide effective agents that can be used for the prevention and treatment of such conditions and diseases that can be treated/prevented by inducing and/or stimulating proteasome degradation, in particular age-related diseases, such as neurological diseases. Other objects and advantages will become apparent to the person of skill when studying the present description of the present invention.

In a first aspect of the present invention, the above object is solved by a molecule improving the degradation of a proteinaceous target molecule by the proteasome of a cell, according to the general formula

B-L-S

wherein B indicates at least one binding moiety binding or promoting binding to the target molecule, S indicates at least one (binding) moiety stimulating proteasome degradation, and L indicates at least one linker moiety suitably linking said at least one binding moiety with said at least one moiety stimulating proteasome degradation, and pharmaceutically acceptable salts thereof.

The molecules as described herein were found to be surprisingly highly effective proteasome inducers/stimulators in order to specifically degrade target molecules. The molecules as described herein were found to have a synergistic effect on the proteasome activity, and/or when degrading the target molecules. Specifically, the molecules provided herein (B-L-S) exhibit the same or even increased stimulation of proteasomal degradation as compared to S alone. Hence, surprisingly and unexpectedly the activity of S is retained or even enhanced by linking it to B with L.

The present inventors thus provide molecules for the induction and/or stimulation of proteasome in diseases and undesired conditions related to an accumulated undesired protein, in particular an accumulated pathological protein in a cell of a mammalian patient or subject, like a human. In the case of a molecule represented by the general structure according to the present invention, the proteasome of the target molecule is effectively induced and/or further stimulated in an ubiquitin independent manner. Preferably, the molecule according to the present invention is bispecific.

The present invention specifically relates to the technology herein designated as TARBOD (TARgeted BOosting Degrader).

Particularly preferred is a molecule according to the present invention that is selected from the group of the compounds according to any one of formulae I to XXVIII, and pharmaceutically acceptable salts thereof.

In a second aspect of the present invention, the above object is solved by a method for producing a molecule according to the present invention, comprising suitably linking the at least one binding moiety (B) to the at least one moiety stimulating proteasome degradation (S) through the at least one linker moiety (L). In a preferred embodiment, the method further comprises the step of identifying the at least one binding moiety (B) and/or the at least one moiety stimulating proteasome degradation (S) before the linking thereof to the at least one linker moiety (L), i.e., involves a pre-selection of suitable components before the chemical and/or enzymatic production of the molecule according to the present invention.

In a third aspect of the present invention, the above object is solved by a pharmaceutical composition, comprising at least one molecule according to the present invention or a pharmaceutically acceptable salt thereof, together with at least one pharmaceutically acceptable carrier or buffer.

In a fourth aspect of the present invention, the above object is solved by the molecule according to the present invention or the pharmaceutical composition according to the present invention for use in the prevention and/or treatment of diseases or conditions in a mammalian subject, such as a human. The diseases or conditions to be treated or prevented are preferably selected from the group consisting of a disease or condition caused by an undesired proteinaceous target molecule, caused by an accumulated pathological protein, proteopathies, alpha-synucleinopathies, tauopathies, neurodegenerative diseases associated with alpha-synuclein accumulation or aggregation, neurodegenerative diseases associated with tau accumulation or aggregation, neurodegenerative diseases associated with beta-amyloid accumulation or aggregation, Parkinson's disease, Alzheimer's disease, dementia, dementia with Lewy bodies, frontotemporal dementia, progressive supranuclear palsy, Pick's disease, amyotrophic lateral sclerosis, Huntington's disease, spinocerebellar ataxias, prion diseases, cystic fibrosis, alpha 1 antitrypsin deficiency, diabetes type-2, retinitis pigmentosa, cataracts, amyloidosis, desmin-related cardiomyopathy, multiple system atrophy, cancer, breast cancer, prostate cancer, cancer tumor metastasis, and ageing.

In a fifth aspect of the present invention, the above object is solved by a method for treating or preventing a disease or condition in a mammalian subject, such as a human, comprising administering to a subject in need of said treatment or prevention an effective amount of the molecule according to the present invention or the pharmaceutical composition according to the present invention, wherein the disease or condition to be treated or prevented is selected from the group consisting of a disease or condition caused by an undesired proteinaceous target molecule, caused by an accumulated pathological protein, proteopathies, alpha-synucleinopathies, tauopathies, neurodegenerative diseases associated with alpha-synuclein accumulation or aggregation, neurodegenerative diseases associated with tau accumulation or aggregation, neurodegenerative diseases associated with beta-amyloid accumulation or aggregation, Parkinson's disease, Alzheimer's disease, dementia, dementia with Lewy bodies, frontotemporal dementia, progressive supranuclear palsy, Pick's disease, amyotrophic lateral sclerosis, Huntington's disease, spinocerebellar ataxias, prion diseases, cystic fibrosis, alpha 1 antitrypsin deficiency, diabetes type-2, retinitis pigmentosa, cataracts, amyloidosis, desmin-related cardiomyopathy, multiple system atrophy, cancer, breast cancer, prostate cancer, cancer tumor metastasis, and ageing.

In a sixth aspect of the present invention, the above object is solved by the use of the molecule according to the present invention or the pharmaceutical composition according to the present invention for improving proteasome function preferably in a mammalian cell, such as a human cell.

In a seventh aspect of the present invention, the above object is solved by the use of the molecule according to the present invention or the pharmaceutical composition according to the present invention for removing an undesired proteinaceous target molecule preferably in a mammalian cell, such as a human cell.

The methods and uses of the present invention can be performed in vivo and/or in vitro.

It was surprisingly found that the molecules according to the present invention act as a proteasome inducer. They are effective and exhibit many advantages compared to other proteasome-related treatment options. In comparison with prior art molecules as tested (e.g. miconazole), an improvement of the present invention lies in the unexpected observation that the molecules as described herein are highly effective proteasome stimulators/inducers (see Examples below). The molecules of the present invention (B-L-S) exhibit the same or even an increased stimulation of proteasomal degradation as compared to the stimulation of proteasomal degradation achieved by S alone. Based on these results it is evident that these molecules will be active in target molecule-related diseases as disclosed herein. Furthermore, specific activities in models of diseases involving or caused by an undesired proteinaceous target molecule, for example caused by an accumulated pathological protein, proteopathies, alpha-synucleinopathies, tauopathies, neurodegenerative diseases associated with alpha-synuclein accumulation or aggregation, neurodegenerative diseases associated with tau accumulation or aggregation, neurodegenerative diseases associated with beta-amyloid accumulation or aggregation, Parkinson's Disease, Alzheimer's Disease, dementia, dementia with Lewy bodies, multiple system atrophy, cancer, breast cancer, prostate cancer, cancer tumor metastasis, and ageing are anticipated. By way of an exemplary application, the molecules according to Formula I to XXVIII were shown to be surprisingly effective, e.g., synergistically effective (see Examples below).

The present invention generally provides molecules that improve—and even boost—the degradation of a proteinaceous target molecule by the proteasome of a cell. According to the invention, a molecule comprises at least the parts B, L, and S that are complexed and/or combined according to the formula B-L-S.

The components as indicated can thus be chemically bound and/or form a complex, i.e., a molecular entity formed by loose association involving two or more of the components based on other physical interactions than covalent bonds, such as charges, atomic interactions, or the like.

Surprisingly, the molecules according to the invention, such as, for example, according to Formula I to XXVIII were shown to be surprisingly effective inducers/stimulators of the proteasome, and even synergistically effective. Without wanting to be bound by theory, it appears that the strong effect is at least in part caused or related to the spatial arrangement of the three components B, L, and S, in particular the rather close molecular distance between B and S, as primarily controlled by the linker L, e.g., in the form of a (PEG)₃₋₅ structure (see examples, in particular Formula I to XXVIII). It seems that the longer the linker (such as a (PEG)₅ structure) the larger is the stimulation.

L indicates at least one linker moiety suitably linking said at least one binding moiety with said at least one moiety stimulating proteasome degradation, and L is preferably selected from the group consisting of a branched or unbranched chemical linker compound, a branched or unbranched linker amino acid sequence, a cleavable branched or unbranched linker compound, and a labelled branched or unbranched linker compound, in particular a polyethylene linker sequence, such as (PEG)₃.

Linkers based on poly(ethylene glycol) (PEG) are the most common types for chemical cross-linking of biomolecules, and therefore are preferred. Alternatives are XTEN, PAS, and GQAP amino acid linkers that are used as “recombinant PEG linkers” for cross-linking biomolecules, via introduction of reactive residues along the sequence (Thomas Kjeldsen, et al. Dually Reactive Long Recombinant Linkers for Bioconjugations as an Alternative to PEG ACS Omega 2020 5 (31), 19827-19833 DOI: 10.1021/acsomega.0c02712; and Podust, V. N.; et al. Extension of in vivo half-life of biologically active molecules by XTEN protein polymers. J. Controlled Release 2016, 240, 52-66, DOI: 10.1016/j.jconrel.2015.10.038). Another alternative is the conjugation with polysarcosine (PSar) (Yali Hu, et al. Polysarcosine as an Alternative to PEG for Therapeutic Protein Conjugation Bioconjugate Chemistry 2018 29 (7), 2232-2238 DOI: 10.1021/acs.bioconjchem.8b00237). A suitable linker is preferably selected based on the size/distance requirements between B and S as mentioned above and should show little or no immunogenicity in vivo. L can be more preferably selected from between (PEG)₂ to (PEG)₅ and branched derivatives thereof.

In one embodiment, L is a linker with a length of about 0.1 nm to about 2.5 nm. In a preferred embodiment, L is a linker with a length of about 0.3 nm to about 1.8 nm. In the context on the molecules of the present invention, the linker serves the spatial arrangement of B and S. This can be achieved by any type of linker exhibiting a suitable linker length. In the context of the present invention, “about” shall include +/−10% of a given value.

In the above formula, B indicates at least one binding moiety binding or promoting binding to the target molecule. Thus, the purpose of B is generally to bind or attach to a target molecule which is then (more efficiently compared to a normal introduction) introduced into the proteasomal degradation process. Target molecules in the context of the present invention are generally any undesired molecules that may be reduced or removed from the cell by proteasomal degradation. Preferably, therefore, target molecules are proteinaceous molecules that relate to or cause an undesired disease or condition, and molecules the removal of which provides a health benefit to the respective subject or patient. More preferred examples of target molecules are selected from an accumulated pathological protein, prions, beta-amyloid, tau, alpha-synuclein, estrogen receptor alpha (ERα), androgen receptor (AR), huntingtin, ataxin, methionine aminopeptidase-1 (MetAP-2), cellular retinoic acid-binding protein-I, cellular retinoic acid-binding protein-II (CRABP-II), alpha 1 antitrypsin, rhodopsin, crystallin, transthyretin, amyloid, cystic fibrosis transmembrane conductance regulator (CTFR), superoxide dismutase 1 (SOD1), transactive response DNA binding protein 43 kDa (TDP-43), fused in sarcoma (FUS), and islet amyloid polypeptide (IAPP), and preferably wherein the target molecule is selected from an extracellular protein.

In the context of the present invention, the at least one binding moiety (B) can be any suitable molecule binding or promoting binding to the target molecule. Respective binding moieties are known to the person of skill, and are disclosed in the literature. Preferred is a binding moiety from the group consisting of an antibody that is specific for the target molecule as above or a specific binding fragment thereof, a proteinaceous group specifically binding to the target molecule, a specifically binding peptide, a natural or non-natural binding nucleic acid, and a small molecule specifically binding to said target molecule, and specific examples are amyvid (florbetapir 18F), tauvid (flortaucipir 18F), and thioflavin.

Binding moieties and linker moieties are e.g. described in Hyun et al. (Chemical-Mediated Targeted Protein Degradation in Neurodegenerative Diseases Life 2021, 11(7), 607). Further examples of suitable binding moieties are provided below, which are known from the prior art:

### Examples of Tau Binding Moieties:

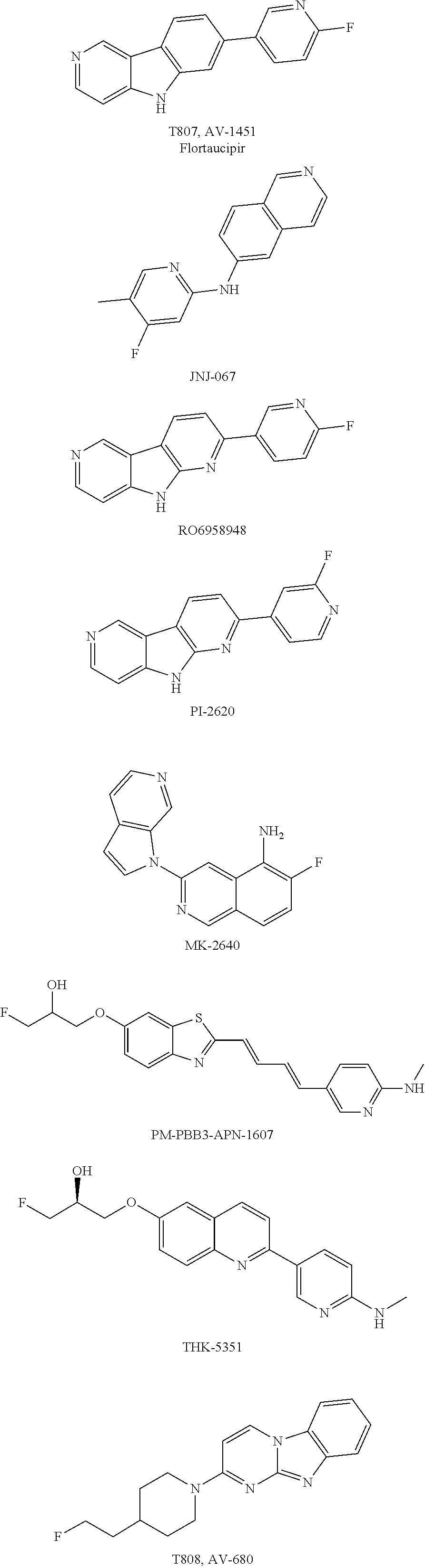

### Examples of α-Synuclein Binding Moieties:

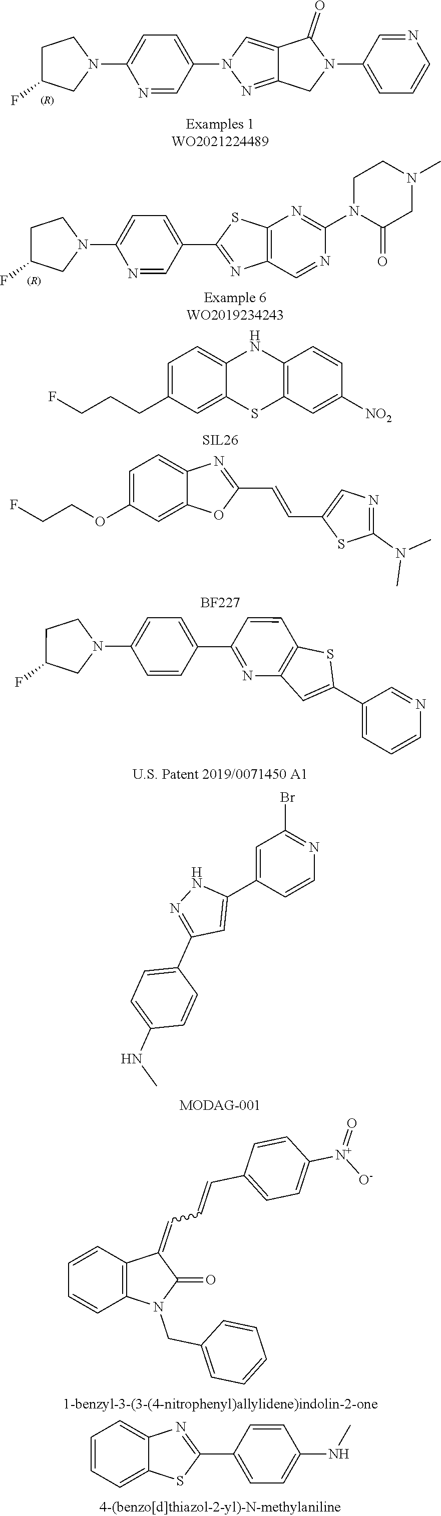

### Examples of Huntingtin Binding Moieties:

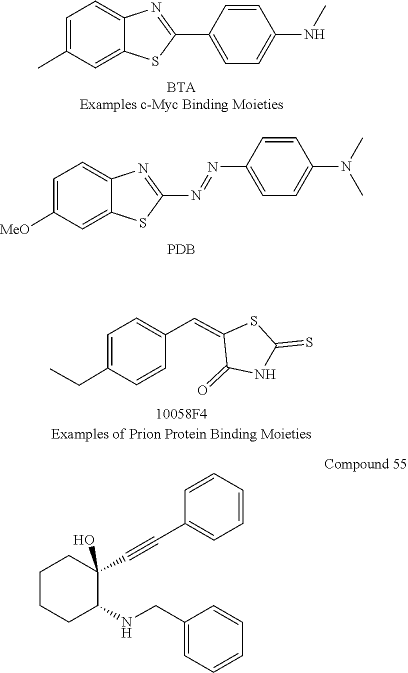

### Examples of Transthyretin (TTR) Binding Moieties:

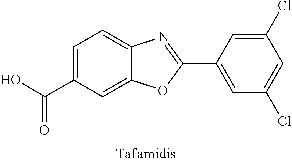 [SMILES: O=C(O)c1ccc2nc(-c3cc(Cl)cc(Cl)c3)oc2c1]

### Examples of DNA Binding Protein-43 (TDP-43) Binding Moieties

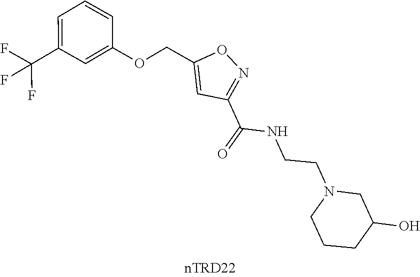 [SMILES: O=C(NCCN1CCCC(O)C1)c1cc(COc2cccc(C(F)(F)F)c2)on1]

The binding moiety B can be fused to the linker moiety L using established methods disclosed in the prior art and in the Examples herein.

In one embodiment, B is a binding moiety as exemplified above or a derivative thereof.

In the context of the present invention, in the above formula, S indicates at least one (binding) moiety stimulating proteasome degradation (S) as an activator/stimulator/enhancer of the 20S proteasome. That is, S (herein also called “stimulator”) binds and enhances/stimulates/activates the proteolytic activity of the 20S proteasome. Respective compounds and moieties are known to the person of skill and are also disclosed in the literature (Trader D J; et al. Establishment of a Suite of Assays That Support the Discovery of Proteasome Stimulators. Biochim. Biophys. Acta, Gen. Subj 2017, 1861, 892-899; and Coleman and Trader RSC Chem. Biol., 2021, 2, 636). The stimulator or moiety stimulating proteasome degradation is selected from the group consisting of at least one compound stimulating degradation in an ubiquitin-independent manner, a compound stimulating the 20S core particle (CP) of the proteasome, such as miconazole (MO), AM-404 (AM), AM-404 derivative TRC-1, and ursolic acid (UA), and further derivatives thereof (see Coleman and Trader RSC Chem. Biol., 2021, 2, 636).

In one embodiment, S is a derivative of miconazole according to the following formula:

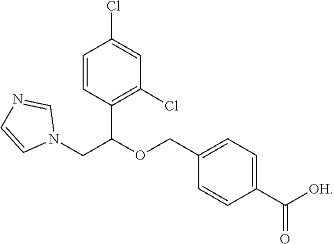 [SMILES: O=C(O)c1ccc(COC(Cn2ccnc2)c2ccc(Cl)cc2Cl)cc1]

Further moieties stimulating the proteasome are disclosed in e.g. WO 2021/034627A1. WO 2021/034627A1 discloses miconazole and miconazole derivatives.

In one embodiment, S is a stimulator of the 20S core particle (CP) of the proteasome as described in any one of claims 1-10 of WO 2021/034627A1. In one embodiment, S is a stimulator of the 20S core particle (CP) of the proteasome selected from MDX1 and MDX2 described in Table 1 of WO 2021/034627A1.

It is intended that the stimulators of the 20S core particle (CP) of the proteasome as described in any one of claims 1-10 and in Table 1 of WO 2021/034627A1 belong to the description of the present invention.

Furthermore, compounds that enhance proteasome degradation in an ubiquitin-dependent manner by binding to the 19S RP can be incorporated into the bifunctional compounds. The proteasome stimulating moiety can have several characteristics, e.g., be a small molecule, or a peptide.

The molecules of the present invention have been shown to stimulate proteasomal activity in a E3 ubiquitin ligase independent manner and in a ubiquitin independent manner in an in vitro setting. Without wishing to be bound by any theory, it is assumed that these molecules also mediate proteasomal degradation of ubiquitinated target proteins in vivo, without, however, requiring such ubiquitination for target degradation.

As mentioned above, the molecules according to the invention were shown to be surprisingly effective inducers/stimulators of the proteasome, and even synergistically effective. Preferred is thus the molecule according to the present invention, wherein said molecule synergistically stimulates/improves the degradation of the proteinaceous target molecule as disclosed herein (see below). In comparison with prior art compounds as tested (including data not shown), an improvement of the present invention lies in the unexpected observation that the compounds described herein are highly effective inducers/stimulators of the proteasome (see Examples below).

The molecules according to the general formula according to the present invention in a preferred embodiment contain miconazole as a proteasome stimulator (Coleman and Trader RSC Chem. Biol., 2021, 2, 636), (PEG)₃ as a linker, and a specific target binder (here, thioflavin, or flortaucipir, or florbetapir). The compounds as described herein are designed to deliver a protein of interest to the proteasome for degradation while boosting proteasome activity.

The molecules according to the general formula according to the present invention can be modified in order to create derivatives of the molecules that are also included in the scope of the present invention. Said modification can take place in an additional preferred step of the methods of the invention as described herein, wherein, for example, after analyzing the degradation of a protein in the presence and absence of said compound as selected, said compound is further chemically modified as described herein, and analyzed again for its effect. Said “round of modification(s)” can be performed for one or several times in all the methods, in order to optimize the effect of the compound, for example, in order to improve its specificity for the target protein. In general, all parts of the molecule may be modified (i.e. B, S), with the linker (L) being less preferred.

This method is also termed “directed evolution” since it involves a multitude of steps including modification and selection, whereby binding compounds are selected in an “evolutionary” process optimizing its capabilities with respect to a particular property, e.g. its binding activity, its ability to activate, inhibit or modulate the activity. The modification can also be simulated in silico before additional tests are performed in order to confirm or validate the effect of the modified selected or screened compound from the first round of screening. Respective software programs are known in the art and readily available for the person of skill.

Modification can further be effected by a variety of methods known in the art, which include without limitation the introduction of novel side chains or the exchange of functional groups like, for example, introduction of halogens, in particular F, Cl or Br, the introduction of lower alkyl groups, preferably having one to five carbon atoms like, for example, methyl, ethyl, n-propyl, isopropyl, n-butyl, isobutyl, tert-butyl, n-pentyl or iso-pentyl groups, lower alkenyl groups, preferably having two to five carbon atoms, lower alkynyl groups, preferably having two to five carbon atoms or through the introduction of, for example, a group selected from the group consisting of NH₂, NO₂, OH, SH, NH, CN, aryl, heteroaryl, COH or COOH group.

The present invention also includes pharmaceutically acceptable salts of the molecules according to the present invention. The term “pharmaceutically acceptable salt” refers to a pharmaceutically acceptable organic or inorganic salt of the compound of the invention. This may include addition salts of inorganic acids such as hydrochloride, hydrobromide, hydroiodide, sulphate, phosphate, diphosphate and nitrate or of organic acids such as acetate, maleate, fumarate, tartrate, succinate, citrate, lactate, methanesulphonate, p-toluenesulphonate, palmoate and stearate. Exemplary salts also include oxalate, chloride, bromide, iodide, bisulphate, acid phosphate, isonicotinate, salicylate, acid citrate, oleate, tannate, pantothenate, bitartrate, ascorbate, gentisinate, gluconate, glucuronate, saccharate, formate, benzoate, glutamate, ethanesulfonate, and benzenesulfonate salts. For other examples of pharmaceutically acceptable salts, reference can be made to Gould (1986, Int J Pharm 33: 201-217).

Most preferred is the molecule according to the present invention, which is selected from the group of the compounds according to any one of formulae I to XXVIII, and pharmaceutically acceptable salts and derivatives thereof as follows.

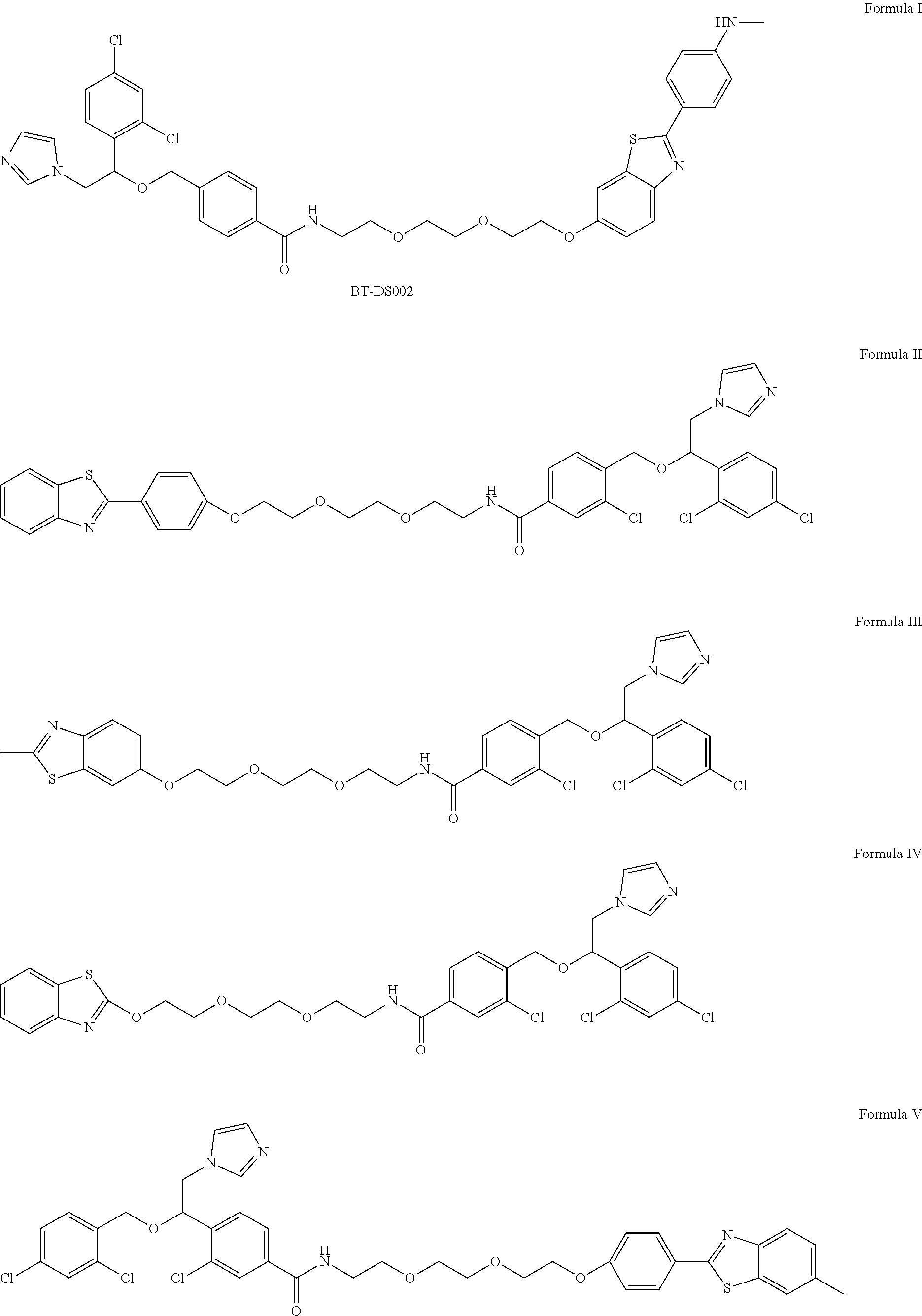

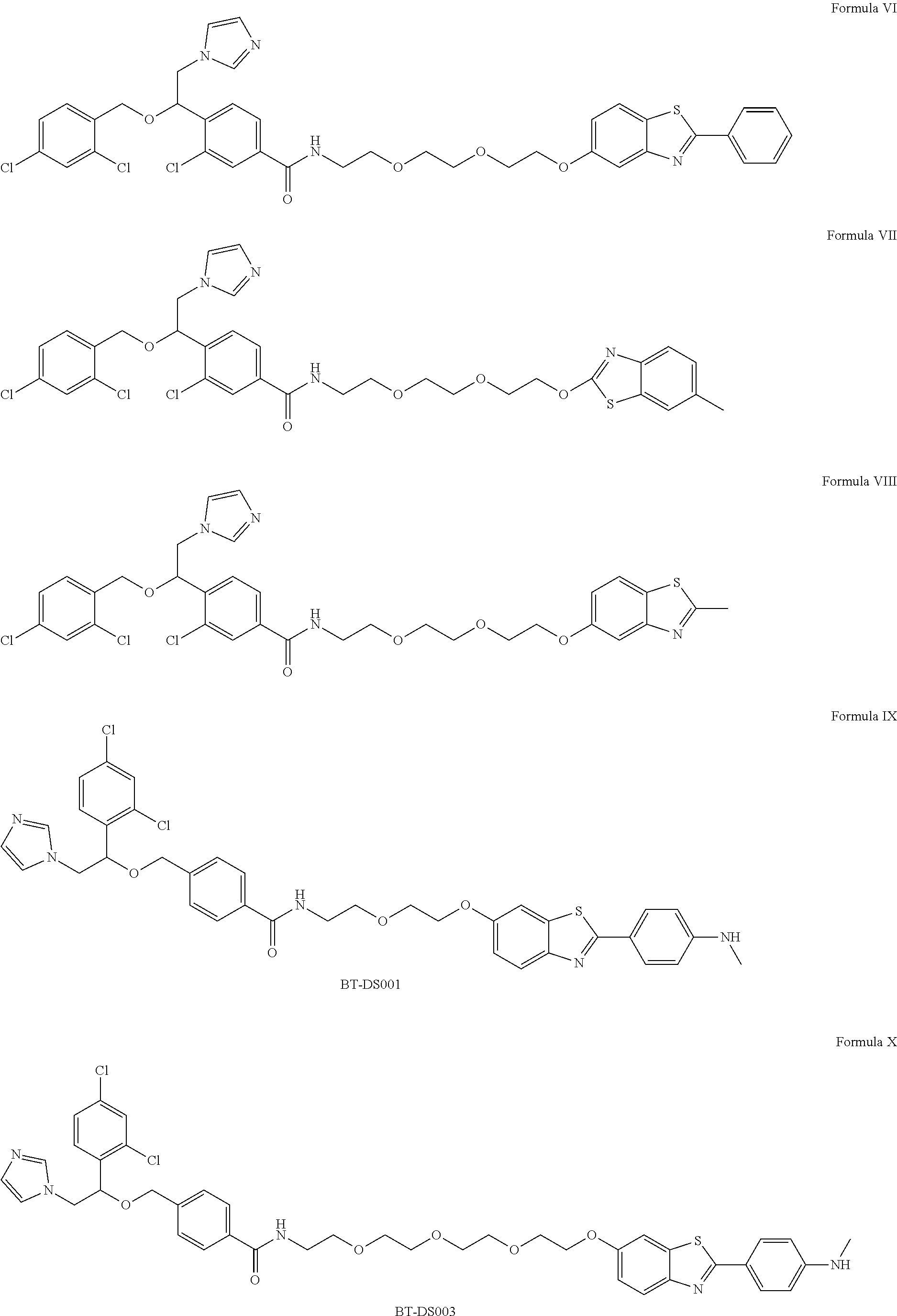

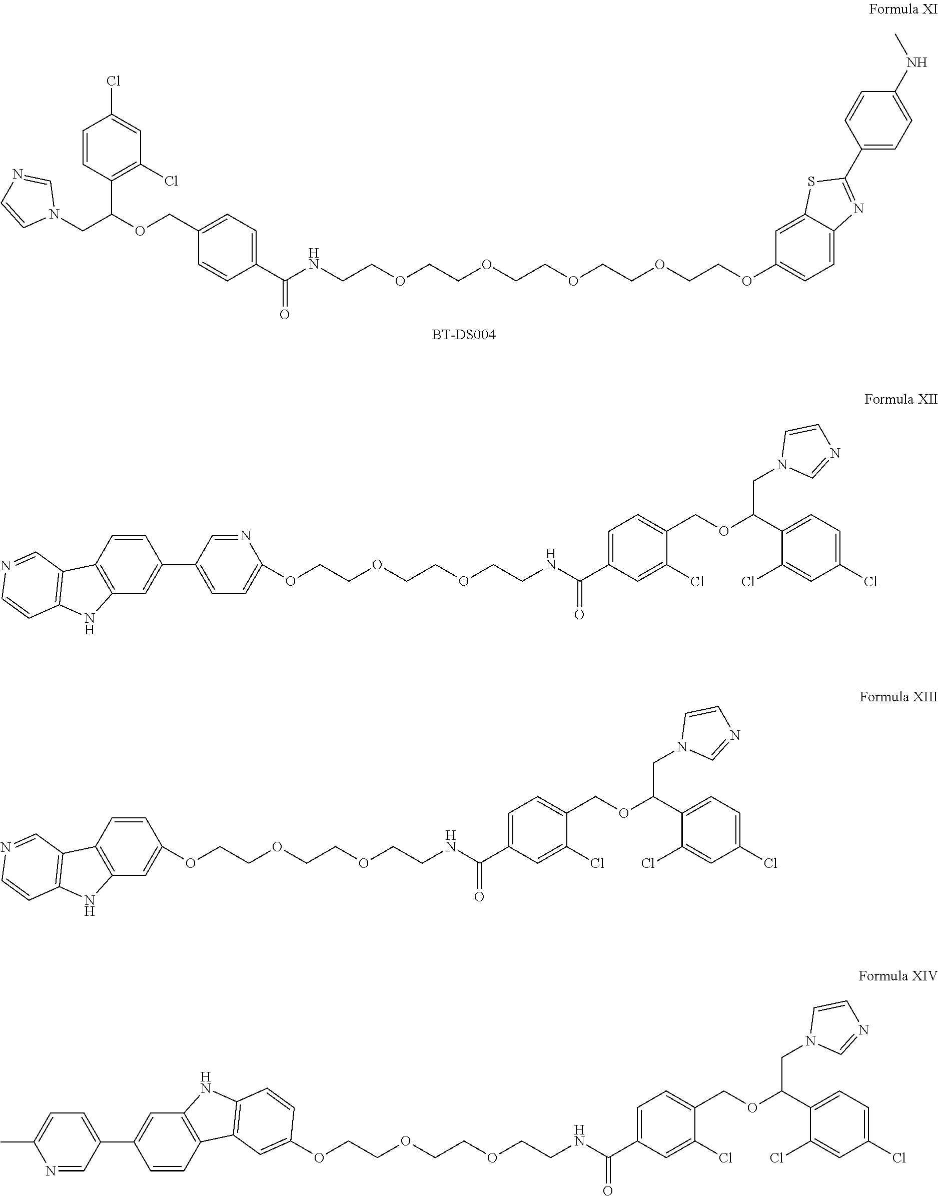

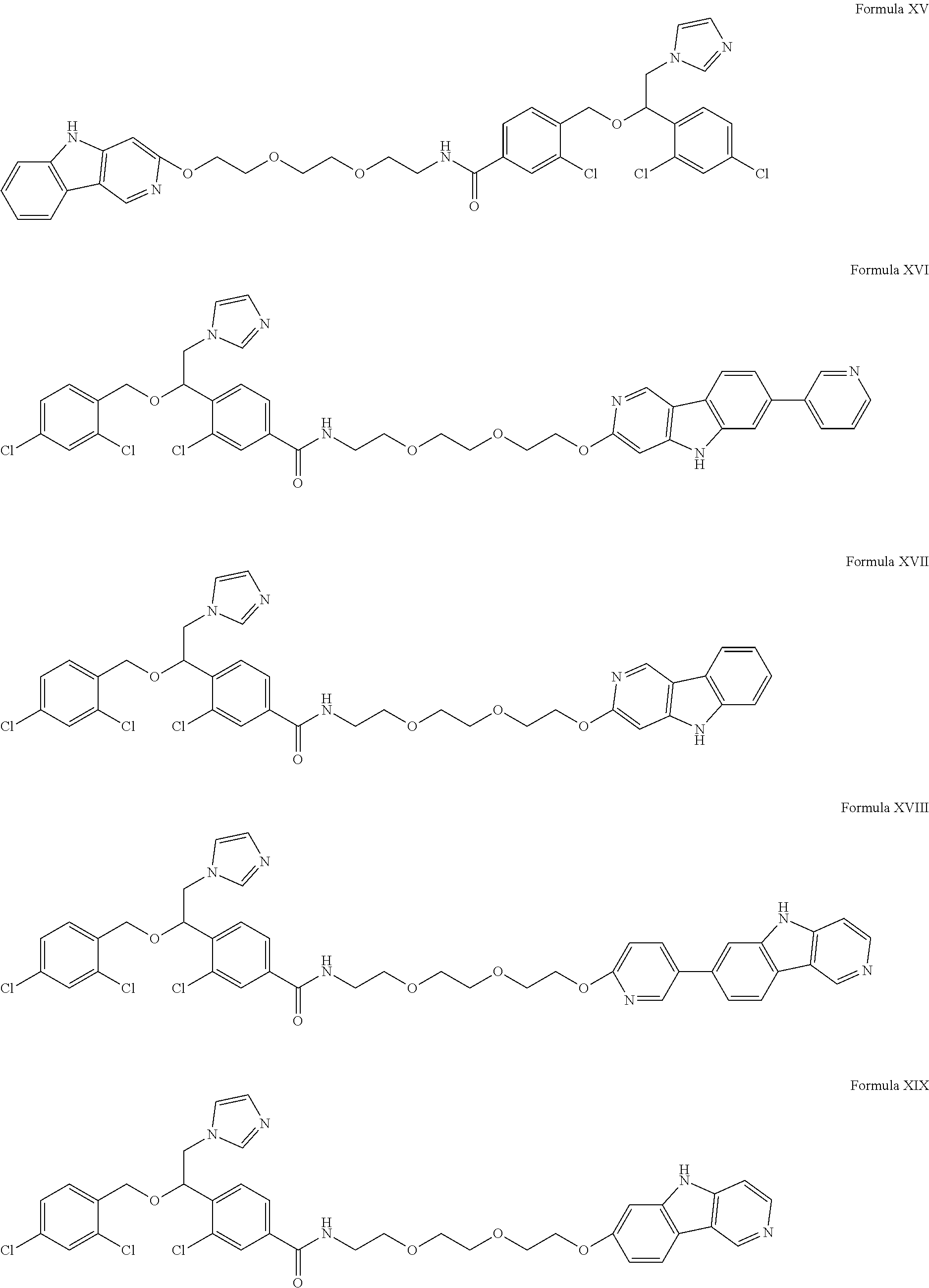 [SMILES: O=C(NCCOCCOCCOc1cc2[nH]c3ccccc3c2cn1)c1ccc(COC(Cn2ccnc2)c2ccc(Cl)cc2Cl)c(Cl)c1 ; O=C(NCCOCCOCCOc1cc2[nH]c3cc(-c4cccnc4)ccc3c2cn1)c1ccc(C(Cn2ccnc2)OCc2ccc(Cl)cc2Cl)c(Cl)c1 ; O=C(NCCOCCOCCOc1cc2[nH]c3ccccc3c2cn1)c1ccc(C(Cn2ccnc2)OCc2ccc(Cl)cc2Cl)c(Cl)c1 ; O=C(NCCOCCOCCOc1ccc(-c2ccc3c(c2)[nH]c2ccncc23)cn1)c1ccc(C(Cn2ccnc2)OCc2ccc(Cl)cc2Cl)c(Cl)c1 ; O=C(NCCOCCOCCOc1ccc2c(c1)[nH]c1ccncc12)c1ccc(C(Cn2ccnc2)OCc2ccc(Cl)cc2Cl)c(Cl)c1]

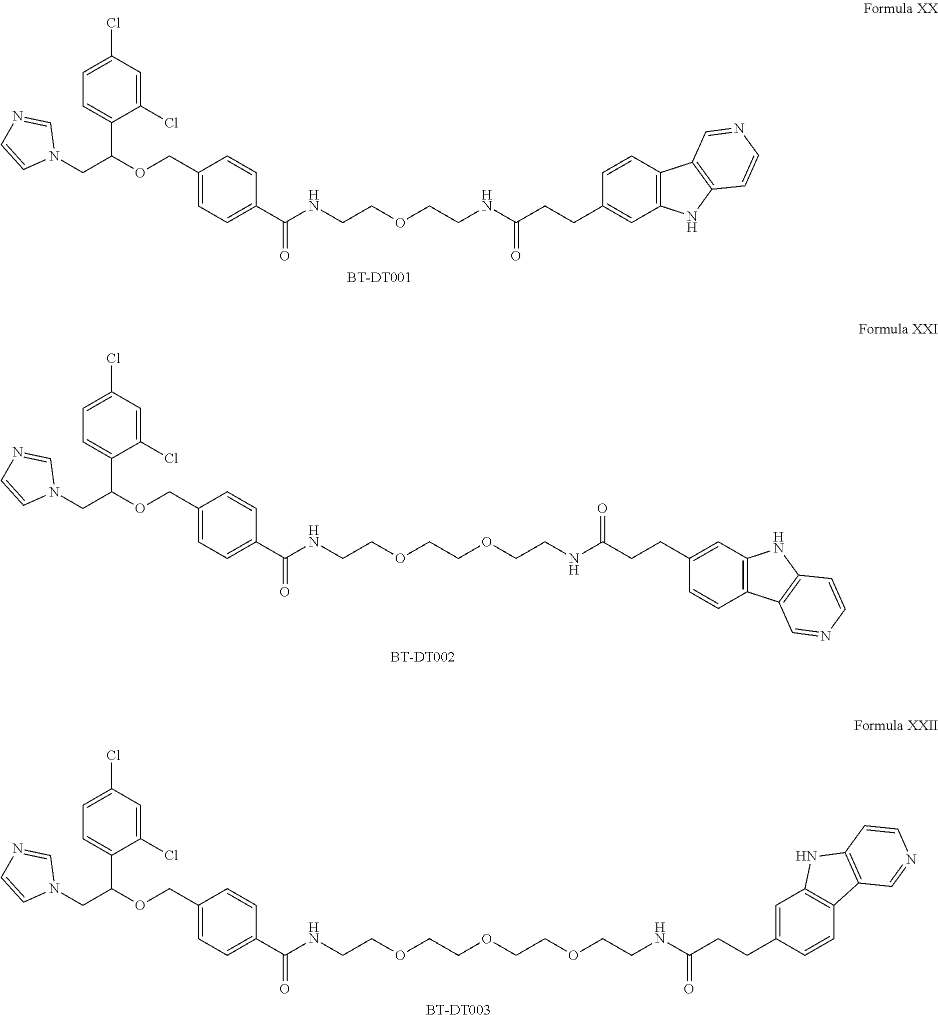

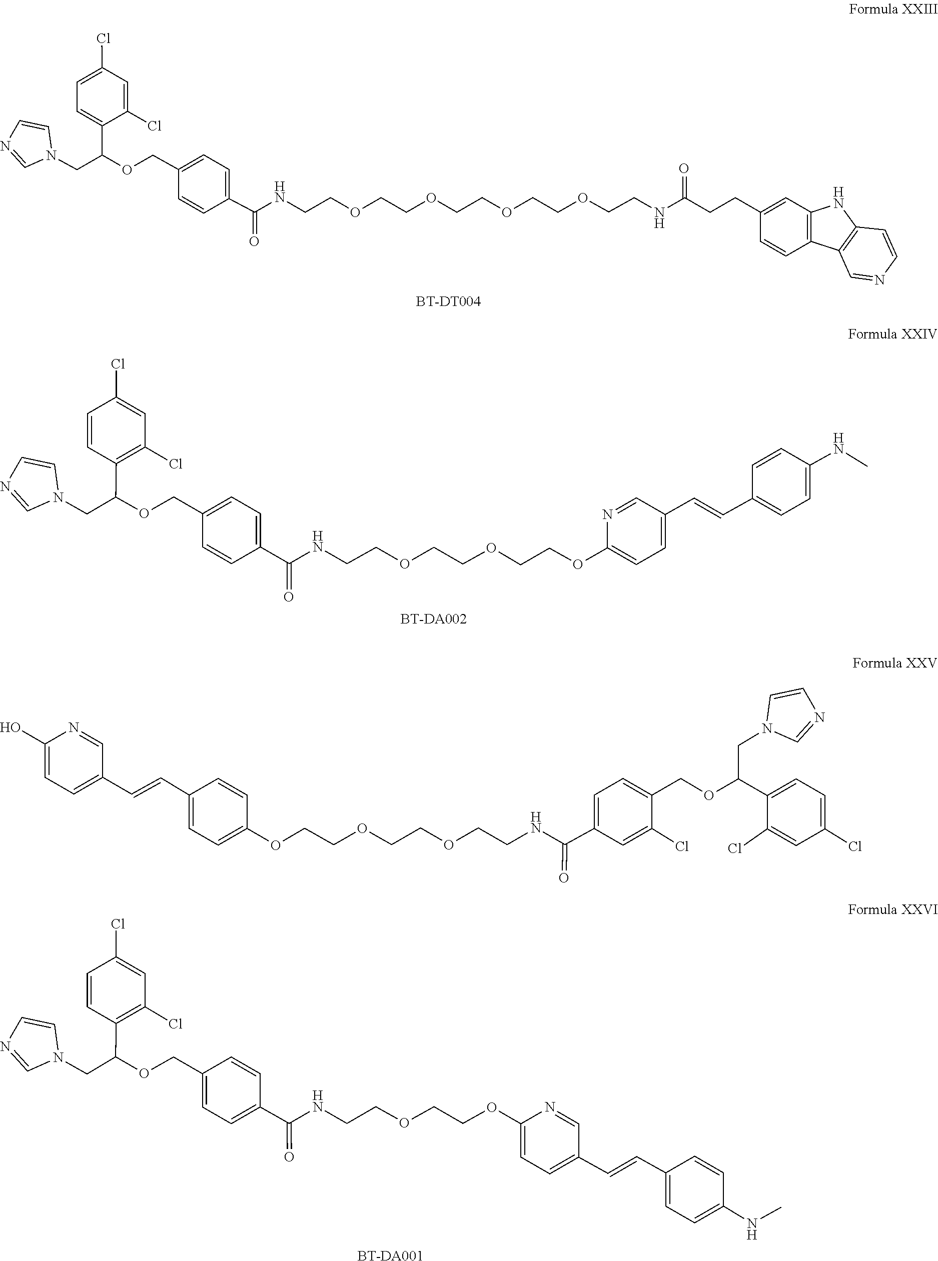

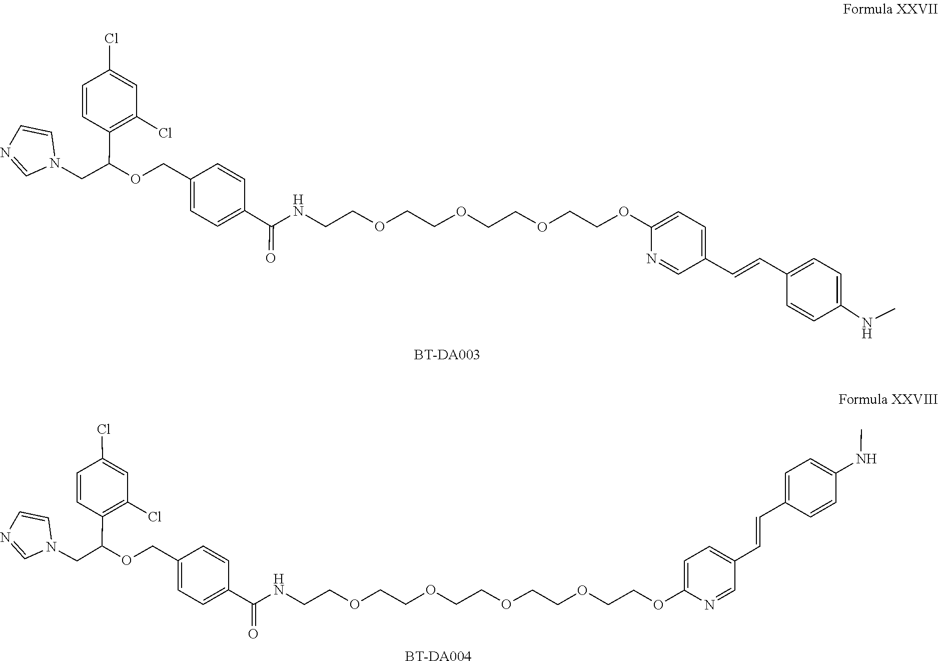

According to a further aspect of the invention, the molecule according to the present invention preferably is bispecific, i.e., has a target molecule binder (B) (or binders to the same target molecule), and a binding moiety stimulating proteasome degradation (S). Nevertheless, in an alternative embodiment the molecule according to the present invention may additionally comprise at least one moiety binding to the proteasome 19S subunit. Accordingly, in these cases L usually is a branched linker molecule.

According to a further aspect of the invention, there is a provided a pharmaceutical composition comprising the molecule according to the present invention, and a pharmaceutically or therapeutically acceptable excipient or carrier. Preferably, the pharmaceutical composition comprises a pharmaceutically effective amount of the molecule according to the present invention.

The term “pharmaceutically or therapeutically acceptable excipient or carrier” refers to a solid or liquid filler, diluent or encapsulating substance which does not interfere with the effectiveness or the biological activity of the active ingredients and which is not toxic to the host, which may be either humans or animals, to which it is administered. Depending upon the particular route of administration, a variety of pharmaceutically-acceptable carriers such as those well known in the art may be used. Non-limiting examples include sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water. Pharmaceutically acceptable carriers or excipients also include diluents (fillers, bulking agents, e.g., lactose, microcrystalline cellulose), disintegrants (e.g., sodium starch glycolate, croscarmellose sodium), binders (e.g. PVP, HPMC), lubricants (e.g., magnesium stearate), glidants (e.g., colloidal SiO₂), solvents/co-solvents (e.g., an aqueous vehicle, Propylene glycol, glycerol), buffering agents (e.g. citrate, gluconates, lactates), preservatives (e.g., Na benzoate, parabens (Me, Pr and Bu), BKC), anti-oxidants (e.g., BHT, BHA, Ascorbic acid), wetting agents (e.g. polysorbates, sorbitan esters), thickening agents (e.g., methylcellulose or hydroxyethylcellulose), sweetening agents (e.g., sorbitol, saccharin, aspartame, acesulfame), flavoring agents (e.g., peppermint, lemon oils, butterscotch, etc.), humectants (e.g., propylene, glycol, glycerol, sorbitol). Other suitable pharmaceutically acceptable excipients are inter alia described in Remington's Pharmaceutical Sciences, 15^{th} Ed., Mack Publishing Co., New Jersey (1991) and Bauer et al., Pharmazeutische Technologic, 5^{th} Ed., Govi-Verlag Frankfurt (1997). The person skilled in the art knows suitable formulations for the compounds according to the present invention, and will readily be able to choose suitable pharmaceutically acceptable carriers or excipients, depending, e.g., on the formulation and administration route of the pharmaceutical composition.

All suitable modes of administration are contemplated according to the invention. For example, administration of the pharmaceutical composition as a medicament may be via oral, subcutaneous, direct intravenous, slow intravenous infusion, continuous intravenous infusion, intravenous or epidural patient controlled analgesia (PCA and PCEA), intramuscular, intrathecal, epidural, intracistemal, intraperitoneal, transdermal, topical, buccal, sublingual, transmucosal, inhalation, intra-atricular, intranasal, rectal or ocular routes, abuse deterrent and abuse resistant formulations, sterile solutions suspensions and depots for parenteral use, and the like, administered as immediate release, sustained release, delayed release, controlled release, extended release and the like. The medicament may be formulated in discrete dosage units and can be prepared by any of the methods well known in the art of pharmacy. The pharmaceutical composition of the present invention can be formulated using methods known in the art to provide rapid, sustained or delayed release of the active ingredient after administration to a mammal.

In addition to the aforementioned molecules of the invention, the pharmaceutical composition can contain two or more molecules according to the present invention and also other therapeutically active substances, depending on the disease or condition to be treated.

The dosage of the pharmaceutical composition according to the present invention can be appropriately selected according to the route of administration, the subject to be administered, the target disease and its severity, age, sex weight, individual differences and disease state. Dosage may be repeated several times a day.

According to the present invention, a subject or patient can be preferably selected from a mammal, such as a mouse, rat, cat, dog, rabbit, goat, sheep, horse, camel, lama, cow, monkey, a farm animal, a sport animal, and a pet, and a human.

According to a further aspect of the invention, there is provided a method for producing the molecule according to the present invention, comprising suitably linking the at least one binding moiety (B) to the at least one moiety stimulating proteasome degradation (S) through the at least one linker moiety (L). Respective chemistry is known to the person of skill, and described in the art and the present examples.

Preferred is a method for producing the molecule according to the present invention, further comprising the step of identifying the at least one binding moiety (B) and/or the at least one moiety stimulating proteasome degradation (S) before the linking thereof to the at least one linker moiety (L). Thus, the method includes a screening step in order to identify suitable binders (e.g., by screening small molecules libraries, DNA-encoded libraries, peptide libraries, fragment-based libraries, antibody libraries) and/or the moiety stimulating proteasome degradation (e.g., by screening small molecules libraries, DNA-encoded libraries, peptide libraries, fragment-based libraries, antibody libraries). Small molecules have been discovered to stimulate the 20S core particle (CP) of the proteasome to degrade proteins, such as a-synuclein (R. A. Coleman and D. J. Trader, A sensitive high-throughput screening method for identifying small molecule stimulators of the core particle of the proteasome, Curr. Protoc. Chem. Biol., 2018, 10(4), e52, DOI: 10.1002/cpch.52.), and the respective screening method(s) can be readily adapted to the present invention. Preferably, a library of small chemical compounds is screened.

A screening step may also be added to the method in order to identify improved molecules according to the invention, where first the molecules is chemically modified, e.g., by adding or deleting chemical groups, such as adding —COOH, and then the activity as well as other pharmacological properties (e.g., stability, half-life or solubility) are tested. Such optimized molecules are then used to produce the molecules of the invention.

Preferably, the methods according to the present invention are amenable to automation, and are preferably performed in an automated and/or high-throughput format. Usually, this involves the use of chips and respective machinery, such as robots. Automation is particularly preferred in case of the identification of binders and stimulators and/or screening.

Further provided is a molecule of the invention as defined herein for use in the prevention and/or treatment of conditions and/or diseases in a mammalian subject, such as a human. A condition and/or disease suitable for treatment according to the relevant aspects of the invention is one which is characterized by the presence and/or accumulation of undesired (usually proteinaceous) molecules that may be reduced or removed from the cell by proteasomal degradation.

The invention further encompasses the use of a molecule of the invention as an inducer/stimulator of proteasomal degradation. The invention further encompasses the use of a molecule of the invention or the pharmaceutical composition of the invention for improving proteasome function. The invention further encompasses the use of a molecule of the invention or the pharmaceutical composition of the invention for removing an undesired proteinaceous target molecule. The uses may be a cosmetic use and/or in vivo and/or in vitro, for example in an in vitro assay.

Modified or altered proteasomal degradation has been shown to be relevant in neurodegenerative disease, as demonstrated by the accumulation of protein aggregates, for example in Alzheimer disease, Parkinson's disease, polyglutamine diseases, and muscular diseases, and amyotrophic lateral sclerosis (ALS).

Therefore, molecules according to the present invention are for use in the prevention and/or treatment of diseases or conditions in a mammalian subject such as a human selected from the group consisting of a disease or condition caused by an undesired proteinaceous target molecule, caused by an accumulated pathological protein, proteopathies, alpha-synucleinopathies, tauopathies, neurodegenerative diseases associated with alpha-synuclein accumulation or aggregation, neurodegenerative diseases associated with tau accumulation or aggregation, neurodegenerative diseases associated with beta-amyloid accumulation or aggregation, Parkinson's disease, Alzheimer's disease, dementia, dementia with Lewy bodies, frontotemporal dementia, progressive supranuclear palsy, Pick's disease, amyotrophic lateral sclerosis, Huntington's disease, spinocerebellar ataxias, prion diseases, cystic fibrosis, alpha 1 antitrypsin deficiency, diabetes type-2, retinitis pigmentosa, cataracts, amyloidosis, desmin-related cardiomyopathy, multiple system atrophy, cancer, breast cancer, prostate cancer, cancer tumor metastasis, and ageing.

By “treatment” or “treating” is meant any treatment of a disease or disorder, in a mammal, including: preventing or protecting against the disease or disorder, that is, causing, the clinical symptoms of the disease not to develop; inhibiting the disease, that is, arresting or suppressing the development of clinical symptoms; and/or relieving the disease, that is, causing the regression of clinical symptoms. By “amelioration” is meant the prevention, reduction or palliation of a state, or improvement of the state of a subject; the amelioration of a stress is the counteracting of the negative aspects of a stress. Amelioration includes, but does not require complete recovery or complete prevention of a stress. Amelioration includes in particular the removal of an undesired protein through proteasomal degradation according to the invention.

Molecules according to the present invention are preferably for use in the prevention and/or treatment of diseases or conditions in a mammalian subject such as a human selected from the group consisting of proteopathies related to the following target molecules (in brackets). Alzheimer's disease (Amyloid, Tau), Frontotemporal Dementia (Tau), Progressive supranuclear palsy (Tau), Pick's disease (Tau), Parkinson's disease (alpha-synuclein), Dementia with Lewy bodies (alpha-synuclein), Multiple system atrophy (alpha-synuclein), Amyotrophic Lateral Sclerosis (SOD1, TDP-43, FUS), Huntington's disease (huntingtin), Spinocerebellar ataxias (ataxin), Prion diseases (prions), Cystic fibrosis (CTFR), Alpha 1 antitrypsin deficiency, (Alpha 1 antitrypsin), Diabetes Type-2 (IAPP), Retinitis Pigmentosa (Rhodopsin), Cataracts (Crystallin), Amyloidosis (Amyloid, Transthyretin), and Desmin-related cardiomyopathy (Crystallin).

The target molecule may be advantageously selected from an extracellular protein, since the 20S proteasome may be located in the circulation (extracellular) of an organism. This use is not available in the context of other molecules, like PROTACs.

Further preferred is the molecule for use according to the present invention, wherein said prevention and/or treatment comprises a combination of at least two molecules for use according to the present invention, and/or a combination with at least one additional pharmaceutically active substance for said undesired protein-related disease or condition. It is to be understood that the present molecule and/or a pharmaceutical composition comprising the present molecule is for use to be administered to a human patient. The term “administering” means administration of a sole therapeutic agent or in combination with another therapeutic agent. It is thus envisaged that the pharmaceutical composition of the present invention is employed in co-therapy approaches, i.e. in co-administration with other medicaments or drugs and/or any other therapeutic agent which might be beneficial in the context of the methods of the present invention. Nevertheless, the other medicaments or drugs and/or any other therapeutic agent can be administered separately from the compound for use, if required, as long as they act in combination (i.e., directly and/or indirectly, preferably synergistically) with the present molecule(s) (for use).

In another aspect thereof, the present invention provides a method for treating or preventing a disease or condition in a mammalian subject, such as a human, comprising administering to a subject in need of said treatment or prevention an effective amount of the molecule according to the present invention or the pharmaceutical composition according to the present invention, wherein the disease or condition to be treated or prevented is selected from the group consisting of a disease or condition caused by an undesired proteinaceous target molecule, caused by an accumulated pathological protein, proteopathies, alpha-synucleinopathies, tauopathies, neurodegenerative diseases associated with alpha-synuclein accumulation or aggregation, neurodegenerative diseases associated with tau accumulation or aggregation, neurodegenerative diseases associated with beta-amyloid accumulation or aggregation, Parkinson's disease, Alzheimer's disease, dementia, dementia with Lewy bodies, frontotemporal dementia, progressive supranuclear palsy, Pick's disease, amyotrophic lateral sclerosis, Huntington's disease, spinocerebellar ataxias, prion diseases, cystic fibrosis, alpha 1 antitrypsin deficiency, diabetes type-2, retinitis pigmentosa, cataracts, amyloidosis, desmin-related cardiomyopathy, multiple system atrophy, cancer, breast cancer, prostate cancer, cancer tumor metastasis, and ageing.

As above for the molecules for use, the dosage of the pharmaceutical composition to be administered according to the present invention can be appropriately selected according to the route of administration, the subject to be administered, the target disease and its severity, age, sex weight, individual differences and disease state. Dosing may be repeated several times a day.

According to the present invention, a mammalian subject or patient can be preferably selected from a mouse, rat, cat, dog, rabbit, goat, sheep, horse, camel, lama, cow, monkey, a farm animal, a sport animal, a pet, and a human.

In addition to the aforementioned molecules of the invention, the pharmaceutical composition as administered can contain two or more molecules according to the present invention and also other therapeutically active substances. In the method, the molecules for use can be provided and/or is administered as a suitable pharmaceutical composition as discussed above. The molecules can be administered alone or in combination with other active molecules or compounds—for example with medicaments already known for the treatment of the aforementioned conditions and/or diseases, whereby in the latter case a favorable additive, amplifying or preferably synergistically effect is noticed.

It was surprisingly found that the molecules according to the invention were shown to be surprisingly effective inducers/stimulators of the proteasome, and even synergistically effective. Preferred is thus the molecule according to the present invention, wherein said molecule synergistically stimulates/improves the degradation of the proteinaceous target molecule as disclosed herein. In comparison with prior art compounds as tested (including data not shown), an improvement of the present invention lies in the unexpected observation that the compounds described herein are highly effective inducers/stimulators of the proteasome (see Examples below).

Without wishing to be bound by any theory, the molecules of the present invention are considered to act by binding to their target protein with the binding moiety B and deliver the target protein to the proteasome for degradation. At the same time, the molecules of the present invention stimulate proteasome activity. Their mode of action is independent of target ubiquitination and, hence, conceptually differs from bifunctional molecules disclosed in the prior art which rely on E3 ubiquitin ligase binding and ubiquitination. In one embodiment, the present invention provides a molecule according to the general formula

B-L-S
wherein
B indicates at least one binding moiety binding or promoting binding to a protein target molecule,
S indicates at least one moiety stimulating proteasomal degradation, and
L indicates at least one linker moiety suitably linking said at least one binding moiety with said at least one moiety stimulating proteasome degradation,
or a pharmaceutically acceptable salt thereof.

In one embodiment, S is a moiety stimulating proteasomal degradation in an E3-ligase independent manner. In one embodiment, S is a moiety stimulating proteasomal degradation in a ubiquitin independent manner. In one embodiment, S is a moiety stimulating proteasomal degradation in an E3-ligase independent manner and in a ubiquitin independent manner.

In one embodiment, S is a moiety stimulating the 20S core particle (CP) of the proteasome.

In one embodiment, S is a moiety stimulating proteasomal activity of the 20S CP of the proteasome by at least 50% over basal activity. In a preferred embodiment, S is a moiety stimulating proteasomal activity of the 20S CP of the proteasome by at least 100% over basal activity.

Proteasomal activity of S may be measured using purified 20S CP of the proteasome, wherein the concentration of the 20S CP of the proteasome is 5 nM and the concentration of S is 0.5, 5 and 25 μM. Incubation of S at the different concentrations with the 20S CP of the proteasome and an activity probe (e.g. labeled protein or artificial substrate) may occur for e.g. 1 h at 37° C. DMSO or any other suitable agent may be used as a control for measuring basal activity. Degradation of the activity probe serves as a readout and may be measured by any suitable method. A stimulation of proteasomal activity by a certain % is achieved in case it is reached for one of the three concentrations of S tested.

In one embodiment, B-L-S stimulates proteasomal activity of the 20S CP of the proteasome by at least 50% over basal activity. In a preferred embodiment, B-L-S stimulates proteasomal activity of the 20S CP of the proteasome by at least 100% over basal activity. In a more preferred embodiment, B-L-S stimulates proteasomal activity of the 20S CP of the proteasome by at least 200% over basal activity.

Proteasomal activity of B-L-S may be measured using purified 20S CP of the proteasome, wherein the concentration of the 20S CP of the proteasome is 5 nM and the concentration of B-L-S is 0.5, 5 and 25 μM. Incubation of B-L-S at the different concentrations with the 20S CP of the proteasome and an activity probe (e.g. labeled protein or artificial substrate) may occur for e.g. 1 h at 37° C. DMSO or any other suitable agent may be used as a control for measuring basal activity. Degradation of the activity probe serves as a readout and may be measured by any suitable method. A stimulation of proteasomal activity by a certain % is achieved in case it is reached for one of the three concentrations of B-L-S tested.

In the context of the above described assays, basal activity of the proteasome is considered to be 100%. For example, stimulation by 50% over basal activity thus refers to 150% activity.

In one embodiment, the molecule of the present invention B-L-S stimulates proteasomal activity of the 20S CP of the proteasome to at least the same degree as S alone. In one embodiment, the molecule of the present invention B-L-S exhibits increased stimulation of proteasomal activity of the 20S CP of the proteasome as compared to S alone. In one embodiment, proteasomal stimulation caused by B-L-S is increased by at least 10%, at least 30%, at least 50% or even at least 100% as compared to the proteasomal stimulation caused by S alone.

Proteasomal activity of B-L-S may be measured using purified 20S CP of the proteasome, wherein the concentration of the 20S CP of the proteasome is 5 nM and the concentration of B-L-S is 0.5, 5 and 25 μM. Incubation of B-L-S with the 20S CP of the proteasome and an activity probe may occur for e.g. 1 h at 37° C. S alone at a concentration 0.5, 5 and 25 μM is used a control for measuring the activity of B-L-S in comparison to S. Degradation of the activity probe serves as a readout and may be measured by any suitable method. A stimulation of proteasomal activity by a certain % is achieved in case it is reached for one of the three concentrations of B-L-S tested as compared to the corresponding concentration of S.

In one embodiment, the 20S CP of the proteasome is the 20S CP of the human proteasome.

In one embodiment, L is a linker with a length of 0.1 nm-2.5 nm. In a preferred embodiment, L is a linker with a length of 0.3 nm-1.8 nm. In the context on the molecules of the present invention, the linker serves the spatial arrangement of B and S. This can be achieved by any type of linker exhibiting a suitable linker length.

The binding moiety binding or the moiety promoting binding to a protein target molecule (B) may be as defined in any of the embodiments provided herein.

The moiety stimulating proteasomal degradation (S) may be as defined in any of the embodiments provided herein.

The present invention will now be described further in the following examples with reference to the accompanying Figures, nevertheless, without being limited thereto. For the purposes of the present invention, all references as cited herein are incorporated by reference in their entireties. In the Figures,

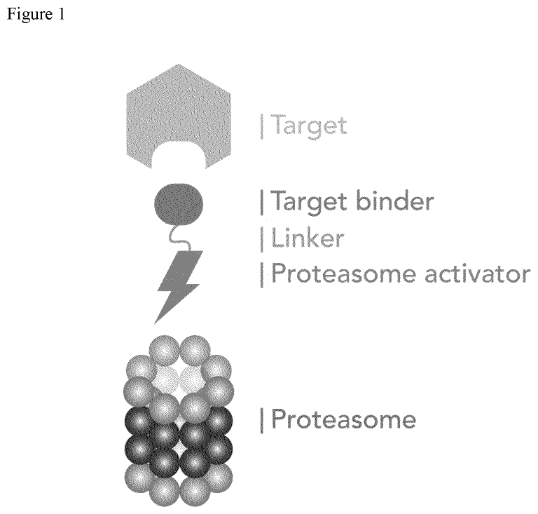

FIG. 1 shows a schematic overview of the function and structure of the molecules according to the present invention.

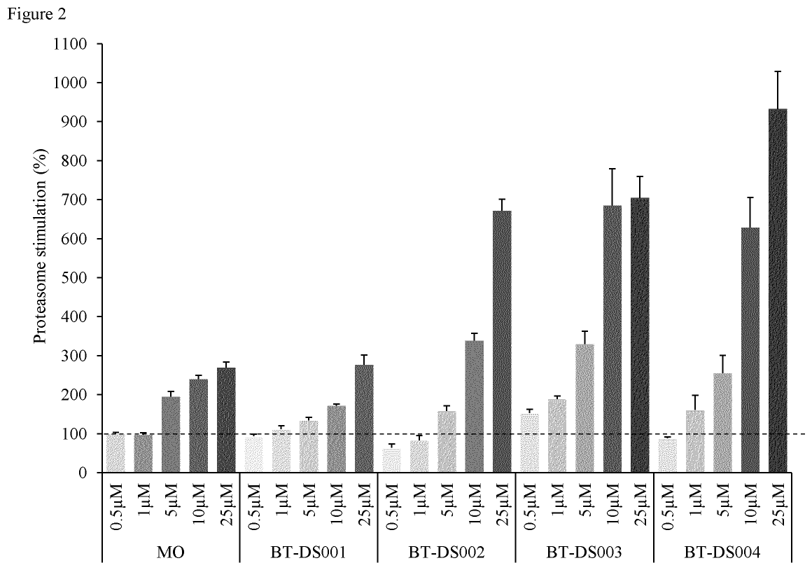

FIG. 2 shows the results of the functional assay for the compounds of group I, based on proteasome stimulation in vitro. The biochemical assay was performed with a purified human 20S proteasome. The compounds were incubated with 20S proteasomes for 1 h, and the proteasome activity was measured with a TAS-1 probe. DMSO samples were used as control (dashed line).

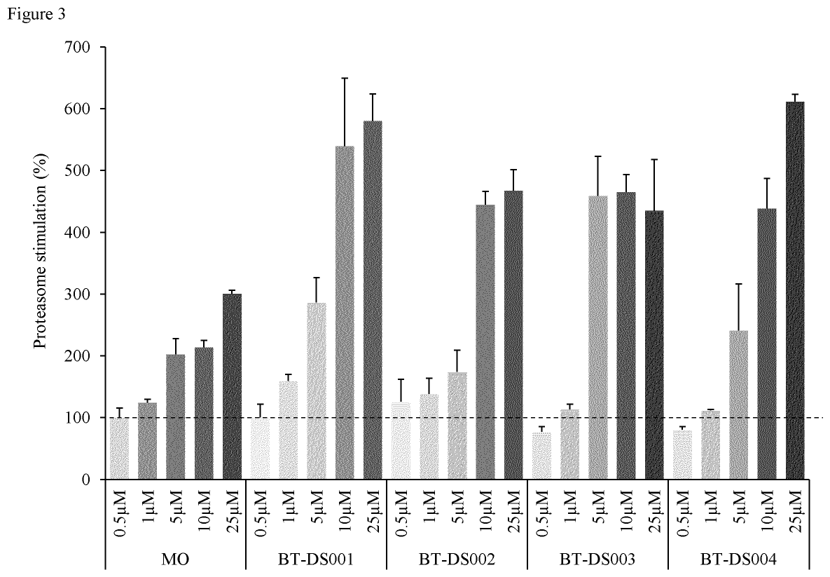

FIG. 3 shows the results of the assay for proteasome activity in HEK293 cells for the compounds of group I. The compounds were dosed for 1.5 h, the proteasome activity was measured with Me4BodipyFLAhx3L3VS activity probe, DMSO samples were used as control (dashed line).

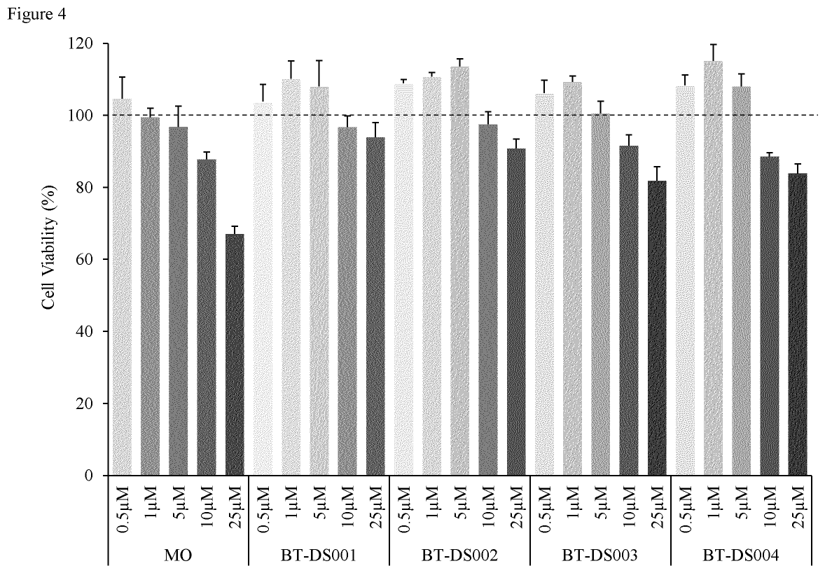

FIG. 4 shows the results of the assay for cellular toxicity in HEK293 cells for the compounds of group I. The compounds were dosed for 24 h, the viability measured with CellTiter-Glo, and DMSO was used as control (dashed line).

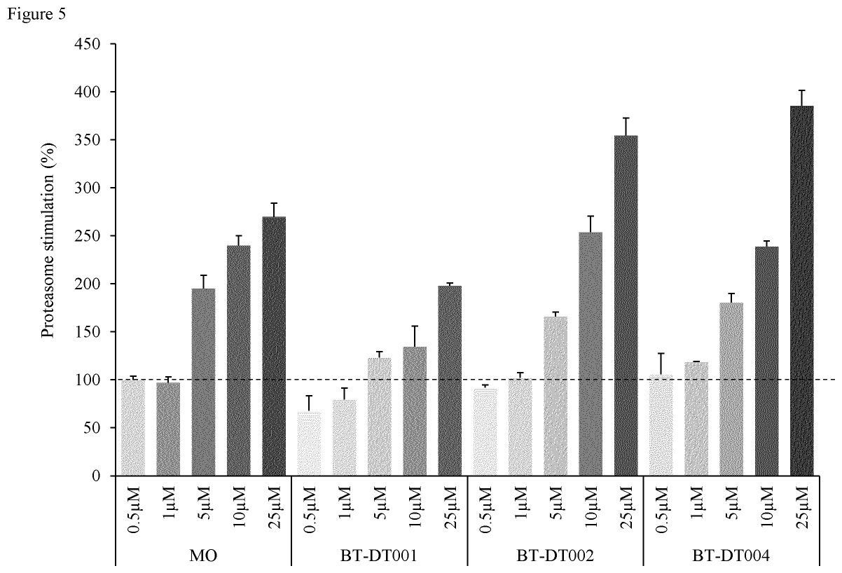

FIG. 5 shows the results of the functional assay for the compounds of group II, based on proteasome stimulation in vitro. The biochemical assay was performed with a purified human 20S proteasome. The compounds were incubated with 20S proteasomes for 1 h, and the proteasome activity was measured with a TAS-1 probe. DMSO samples were used as control (dashed line).

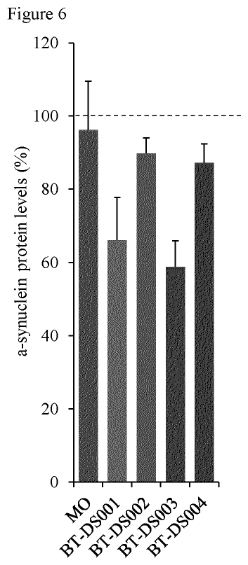

FIG. 6 shows the results of the functional assay for the compounds of group I, a-synuclein degradation in HEK293 cells. After a-synuclein transfection for 48 h, the compounds were dosed for 3 h at 25 μM together with 50 μM cycloheximide, and the total a-synuclein was determined using western blot. DMSO samples were used as control (dashed line).

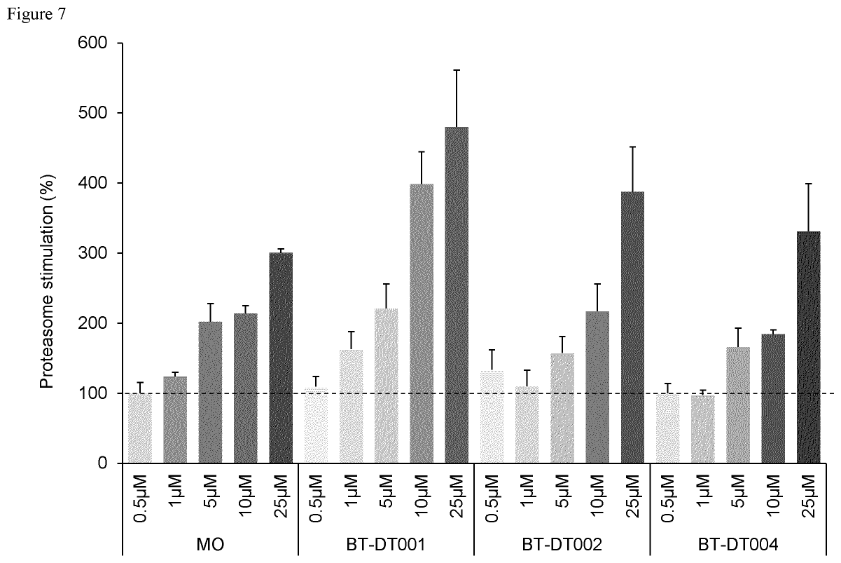

FIG. 7 shows the results of the functional assay for the compounds of group II, proteasome stimulation in cells. For proteasome activity in HEK293 cells, the compounds were dosed for 1.5 h, and the proteasome activity measured with Me4BodipyFLAhx3L3VS activity probe. DMSO samples were used as control (dashed line).

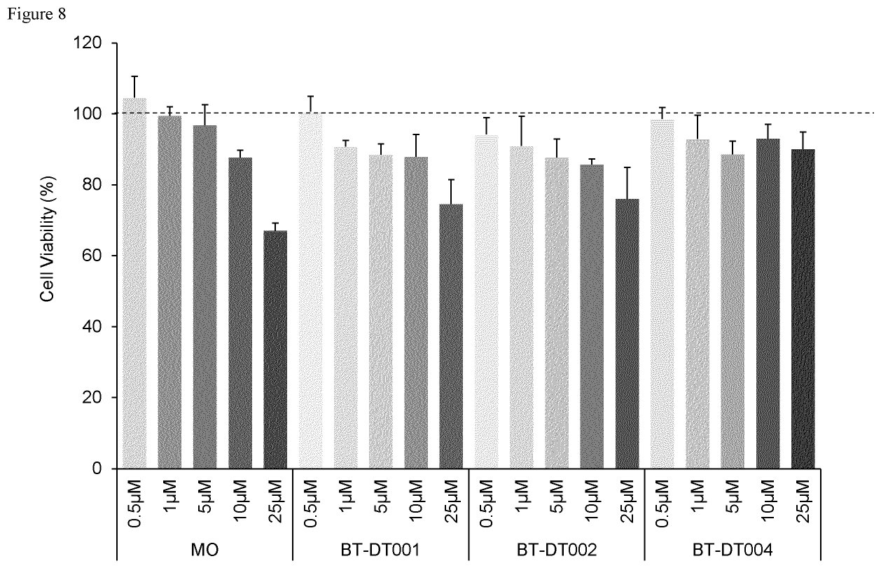

FIG. 8 shows the results of the cell toxicity assay in HEK293 cells for the compounds of group II. Compounds were dosed for 24 h, and the viability measured with CellTiter-Glo. DMSO samples were used as control (dashed line).

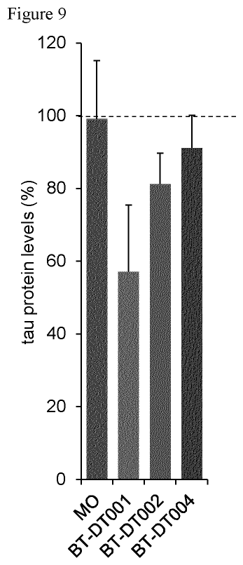

FIG. 9 shows the results of the functional assay for the compounds of group II, tau degradation in HEK293 cells. After tau transfection for 48 h, the compounds were dosed for 16 h at 25 μM together with 30 μM cycloheximide, and total tau was determined (western blot). DMSO samples were used as control (dashed line). Preferred compound DT-001 shows clear tau degradation improvement.

### EXAMPLES

In the context of the present invention, the following molecules were produced (synthesized), and tested. The molecules as a preferred embodiment comprise miconazole as a proteasome stimulator (Trader D J, RSC Chemical Biology, 2021), (PEG)ₓ with x selected from 3 to 5 as a preferred linker, and a specific target binder (here thioflavin, or flortaucipir, or florbetapir). The compounds here described are designed to deliver a protein of interest to the proteasome for degradation while boosting proteasome activity.

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Compounds of the present disclosure may be prepared by methods known in the art of organic synthesis. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (2014) Protective Groups in Organic Synthesis, 5th edition, John Wiley & Sons). These groups are removed at a convenient stage of the compound synthesis using methods that are readily apparent to those skilled in the art.

Unless otherwise stated, all reagents and solvents were obtained from commercial sources and used without further purification. All starting materials, building blocks, reagents, acids, bases, dehydrating agents, solvents, and catalysts utilized to synthesis the compounds of the present invention are either commercially available or can be produced by organic synthesis methods known to one of ordinary skill in the art. The chemical names were generated using ChemBioDraw Ultra v 20.1 from CambridgeSoft.

Temperatures are given in degrees Celsius. If not mentioned otherwise, all evaporations are performed under reduced pressure, typically between about 15 mm Hg and 100 mm Hg (=20-133 mbar).

Abbreviations used are those conventional in the art.

### Analytical Details

All moisture-sensitive reactions were conducted in oven-dried glassware under an argon atmosphere. TLC analysis was conducted using glass-backed thin-layer silica gel chromatography plates (60 Å, 250 μm thickness, F-254 indicator). HPLC data was obtained using both, a preparative Agilent HPLC system (Agilent Technologies 1200 Series) in a reversed phase column reversed phase column (Agilent Eclipse XDB-C18) and an analytical Agilent HPLC system (Agilent 1260 Infinity system, Eclipse Plus C18 m, 4.6×150 mm column). Samples were detected using a UV lamp at 210 nm. Identity and purity of each synthesized molecule was confirmed by LC/MS (Agilent 1260 Infinity II with a Zorbax Eclipse Plus C18 column, 2.1×50 mm, 3.5-micron attached to an Agilent 6129 quadrupole mass spectrometer). Chromatography was performed using silica gel (Fluka: Silica ge101 60, 0.063-0.2 mm) and suitable solvents as indicated in specific examples.

### I. A-Synuclein Degradation Molecules

The molecules of this group are composed of miconazole as the activator, (PEG)x as the ligand, and thioflavin as well as derivatives thereof as the target binder. These compounds are thus designed to promote the degradation of a-synuclein with enhanced proteasome activity.

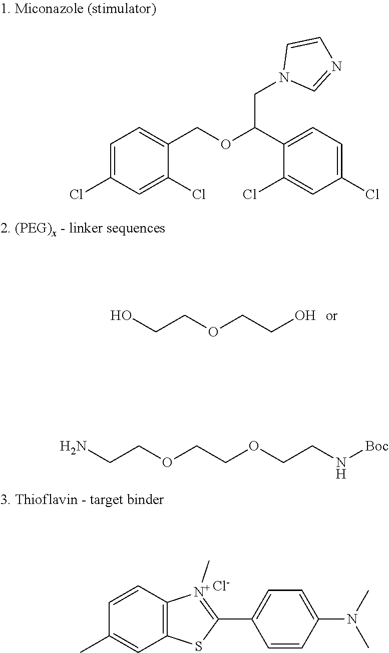

Thioflavins (ThT) are fluorescent dyes used to monitor protein aggregation binding to monomers, oligomers, and fibrils (from a-synuclein), prevent protein aggregation, and extend lifespan (Alavez, S., Vantipalli, M., Zucker, D. et al. Amyloid-binding compounds maintain protein homeostasis during ageing and extend lifespan. Nature 472, 226-229 (2011). https://doi.org/10.1038/nature09873; Romani M, Sorrentino V, Oh C M, Li H, de Lima T I, Zhang H, Shong M, Auwerx J. NAD⁺ boosting reduces age-associated amyloidosis and restores mitochondrial homeostasis in muscle. Cell Rep. 2021 Jan. 19; 34(3):108660. doi: 10.1016/j.celrep.2020.108660. PMID: 33472069; PMCID: PMC7816122).

### Group I: Compounds According to the Present Invention

The compounds of Group I were generally synthesized as follows:

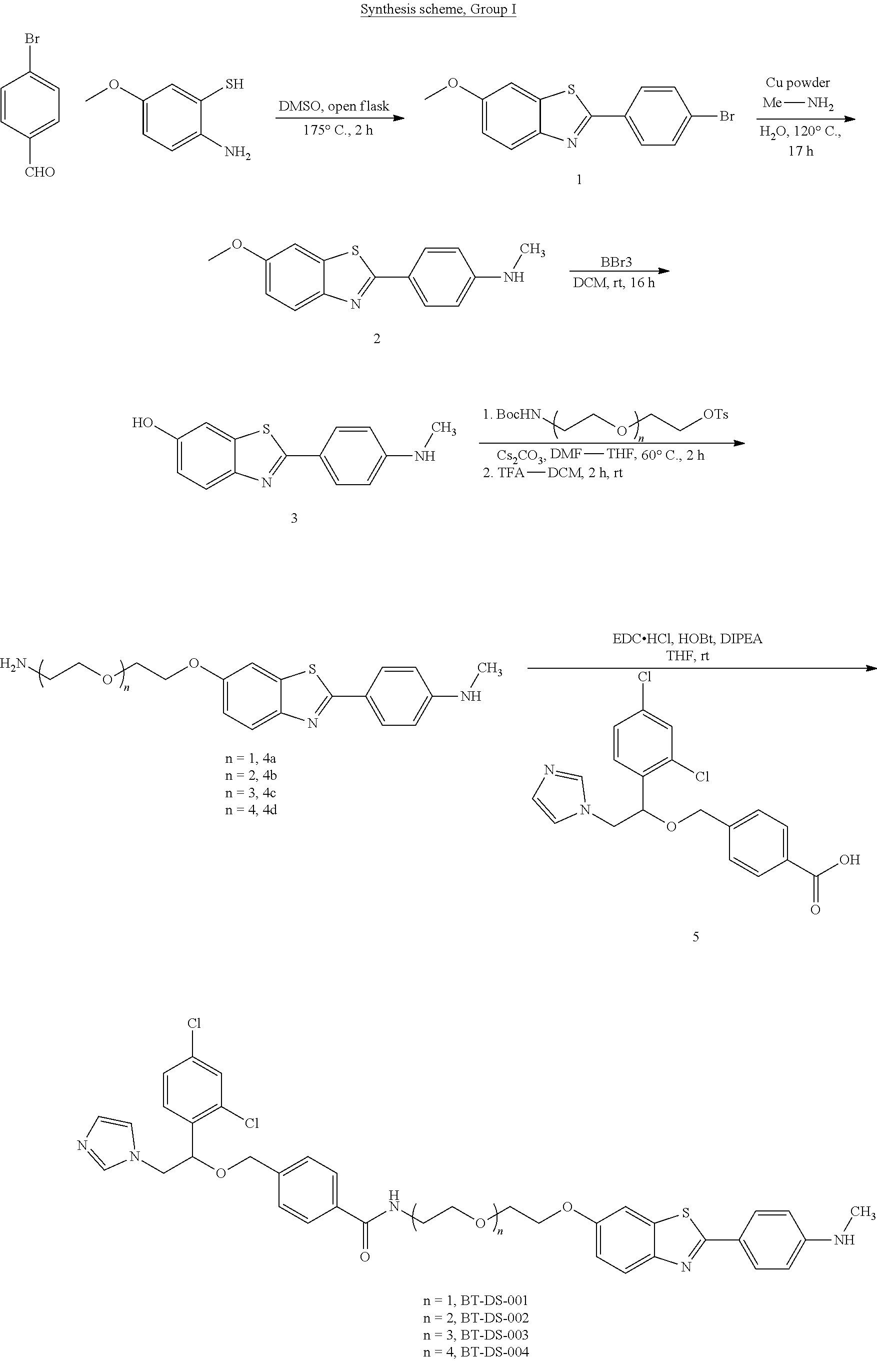

### Compound 1: 2-(4-bromophenyl)-6-methoxybenzo[d]thiazole

A 50 ml round bottom flask was charged with 4-bromobenzaldehyde (2 g, 12.89 mmol) and 2-amino-5-methoxybenzenethiol (2.38 g, 12.89 mmol) followed by DMSO (12 mL). The suspension was briefly bubbled with air, then heated to 175° C. open to the air and stirred for 2 h. After completion of the reaction, the mixture was poured into ice water to precipitate the product, which was collected by suction filtration and washed with water to remove DMSO to afford the title compound (4 g, 96%) as a solid. The solid was used for the next step without further purification.

### Compound 2: 4-(6-methoxybenzo[d]thiazol-2-yl)-N-methylaniline

A mixture of 2-(4-bromophenyl)-6-methoxybenzo[d]thiazole (1 g, 3.12 mmol), 30% aqueous methylamine solution (5 eq), copper powder (20 mg, 0.2 eq) was sealed in a 30 mL screwed tube and stirred in an oil bath at 120° C. for 17 hrs. During the reaction, most of the copper powder was dissolved. The reaction mixture was then cooled to room temperature and ethyl acetate (20 mL) was added to extract the aryl amine. The organic layer was separated, and the aqueous layer was extracted twice with ethyl acetate. The combined extracts were dried by anhydrous sodium sulfate and the solvent was removed under reduced pressure to give the crude product that was purified by silica gel column chromatography (eluent: hexane/ethyl acetate) to afford the title compound.

### Compound 3: 2-(4-(methylamino)phenyl)benzo[d]thiazol-6-ol

To a suspension of 4-(6-methoxybenzo[d]thiazol-2-yl)-N-methylaniline (200 mg, 0.7 mmol) in anhydrous methylene chloride (20 mL) under argon was injected BBr₃ (1 M in DCM, 2.3 mL, 2.3 mmol). The reaction mixture was stirred at room temperature for 16 h. The reaction was quenched with water and the pH was adjusted to 7-8 with NaOH solution and extracted with DCM. The combined extracts were dried with anhydrous sodium sulfate and the solvent was removed under reduced pressure to afford the title compound as a brown solid (70 mg, 36%). The crude was used without further purification.

### Compound 4a-d: General Procedure for the Synthesis of PEGylated Benzothiazoles

A 25 ml round bottom flask under argon was charged with 2-(4-(methylamino)phenyl)benzo[d]thiazol-6-ol (1 eq), BocNH-(PEG)n-CH2CH2OTs (1.2 eq), Cs₂CO₃ (3 eq) in THF-DMF (4:1 v/v), and heated at 60° C. for 2 hrs or until starting material was consumed. The solvent was removed in vacuo, washed with water, and extracted with ethyl acetate. The combined extracts were dried with anhydrous sodium sulfate and the solvent was removed under reduced pressure and the crude was purified by silica gel column chromatography (eluent: DCM/MeOH 95/5). The product was then dissolved in a mixture of TFA and DCM (50/50) and stirred for 2 hrs. DCM was added to the reaction mixture followed by a saturated solution of NaHCO₃ to neutralize TFA. The aqueous layer was extracted twice using DCM and the combined extracts were dried using anhydrous sodium sulfate, filtered and the solvent was removed under reduced pressure to afford the respective Boc deprotected product 4 (a-d), which was used without further purification.

### Compound 5: 4-((1-(2,4-dichlorophenyl)-2-(1H-imidazol-1-yl)ethoxy)methyl)benzoic acid

A flame dried 500 ml round bottom flask was charged with 1-(2,4-dichlorophenyl)-2-(1H-imidazol-1-yl)ethan-1-ol (5 g, 19.45 mmol) under argon atmosphere. Anhydrous THF (65 ml) was added to the flask followed by 10 ml of anhydrous DMF. The mixture was stirred and cooled to 0° C. using an ice bath. After stirring for 5 min, NaH (3 eq, 60% emulsion) was added in portions to the flask under positive pressure of argon. After all NaH was added, the reaction mixture was allowed to stir for 45 min in the ice bath; then the ice bath was removed, and the reaction mixture was stirred for an additional 15 min. The round bottom flask was again put in ice bath, and methyl 4-(bromomethyl)benzoate (1.05 eq, 4.68 g) dissolved in an anhydrous THF (10 ml) was added to the reaction mixture dropwise over 30 min. The reaction mixture was left to stir overnight at room temperature. After completion, the reaction was quenched with a solution of saturated NH₄Cl, and the aqueous layer was extracted with DCM. The combined extracts were dried by anhydrous sodium sulfate and the solvent was removed under reduced pressure. Residual DMF was removed under high vacuum. THF (30 ml) was added to the above crude followed by 30 ml aq. LiOH (5 eq) dropwise. The reaction mixture was stirred for 2 hr until completion. After completion, THF was removed in vacuo, and the pH was adjusted to 5.5 at which point the product precipitated as a white solid. The product was filtered using gravity or vacuum filtration and washed with sat. NH₄Cl. The crude product was recrystallized from H₂O-EtOH to afford the title compound.

### Compound BT-DS-(001-004): General Procedure of Amide Coupling

In a 8 ml vial was added 4-((1-(2,4-dichlorophenyl)-2-(1H-imidazol-1-yl)ethoxy)methyl)benzoic acid (10 mg, 1 eq), HOBt hydrate (2 eq), EDC hydrochloride (1.2 eq) followed by DCM (1 ml) and DIPEA (4 eq). The contents were stirred for 5 min and then 4 (a-d) (1.1 eq) was added to the vial. The reaction mixture was stirred until completion and a saturated solution of NH₄Cl was added to the reaction mixture. The aqueous layer was extracted 3 times using DCM; the combined extracts were dried using anhydrous sodium sulfate, filtered and the solvent was removed under reduced pressure. The crude was purified on reversed phase HPLC using water-acetonitrile (with 0.1% TFA). The lyophilized product (TFA salt of product) was neutralized using a saturated solution of NaHCO₃ and extracted using DCM or ethyl acetate. The combined extracts were dried using sodium sulfate, filtered and the solvent was removed under reduced pressure to afford the title compound.

Following the Amide coupling procedure as described above, the following compounds were prepared.

| Examples | Product/Mass |
|---|---|
| BT-DS001 4-((1-(2,4-dichlorophenyl)- 2-(1H-imidazol-1- yl)ethoxy)methyl)-N-(2-(2- ((2-(4- (methylamino)phenyl)benzo [d]thiazol-6- yl)oxy)ethoxy)ethyl) benzamide | 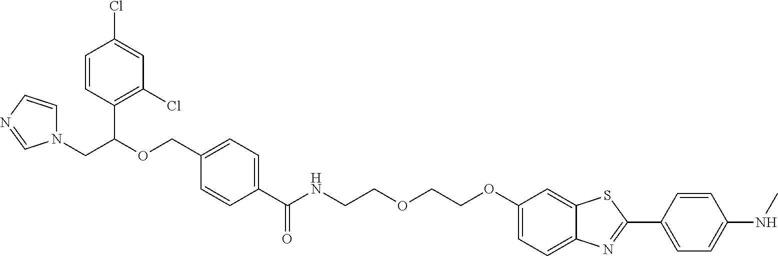 [SMILES: CNc1ccc(-c2nc3ccc(OCCOCCNC(=O)c4ccc(COC(Cn5ccnc5)c5ccc(Cl)cc5Cl)cc4)cc3s2)cc1] |
| | 716.2 (M + H+) |
| | 358.7 (M + 2H+)/2 |
| BT-DS002 4-((1-(2,4-dichlorophenyl)- 2-(1H-imidazol-1- yl)ethoxy)methyl)-N-(2-(2- (2-((2-(4- (methylamino)phenyl)benzo [d]thiazol-6- yl)oxy)ethoxy)ethoxy)ethyl) benzamide | 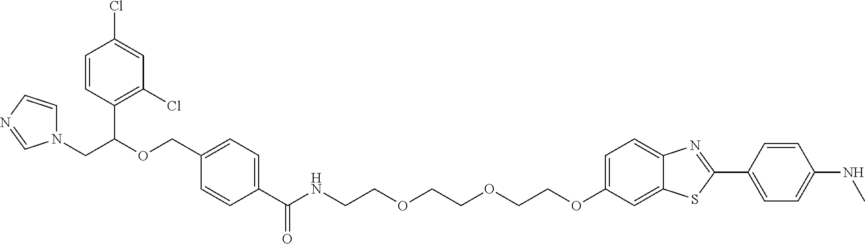 [SMILES: CNc1ccc(-c2nc3ccc(OCCOCCOCCNC(=O)c4ccc(COC(Cn5ccnc5)c5ccc(Cl)cc5Cl)cc4)cc3s2)cc1] |
| | 760.2 (M + H+) |
| | 380.7 (M + 2H+)/2 |
| BT-DS003 4-((1-(2,4-dichlorophenyl)- 2-(1H-imidazol-1- yl)ethoxy)methyl)-N-(2-(2- (2-(2-((2-(4- (methylamino)phenyl)benzo [d]thiazol-6- yl)oxy)ethoxy)ethoxy) ethoxy)ethyl)benzamide | 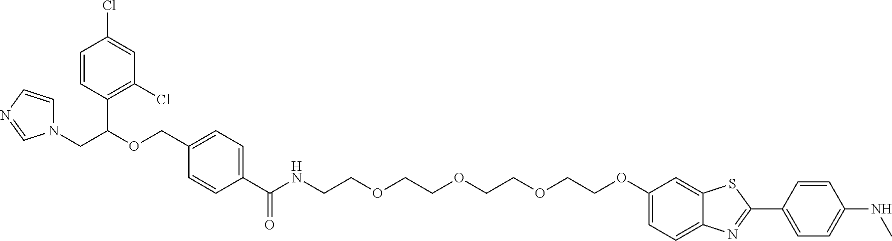 [SMILES: CNc1ccc(-c2nc3ccc(OCCOCCOCCOCCNC(=O)c4ccc(COC(Cn5ccnc5)c5ccc(Cl)cc5Cl)cc4)cc3s2)cc1] |
| | 804.4 (M + H+) |
| | 402.7 (M + 2H+)/2 |
| BT-DS004 4-((1-(2,4-dichlorophenyl)- 2-(1H-imidazol-1- yl)ethoxy)methyl)-N-(14- ((2-(4- (methylamino)phenyl)benzo [d]thiazol-6-yl)oxy)- 3,6,9,12- tetraoxatetradecyl)benzamide | 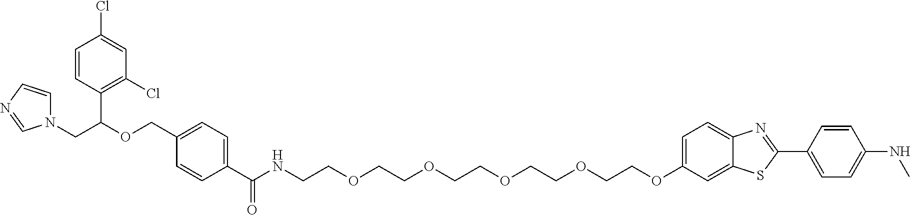 [SMILES: CNc1ccc(-c2nc3ccc(OCCOCCOCCOCCOCCNC(=O)c4ccc(COC(Cn5ccnc5)c5ccc(Cl)cc5Cl)cc4)cc3s2)cc1] |
| | 848.2 (M + H+) |
| | 424.8 (M + 2H+)/2 |

### Compounds Group I:

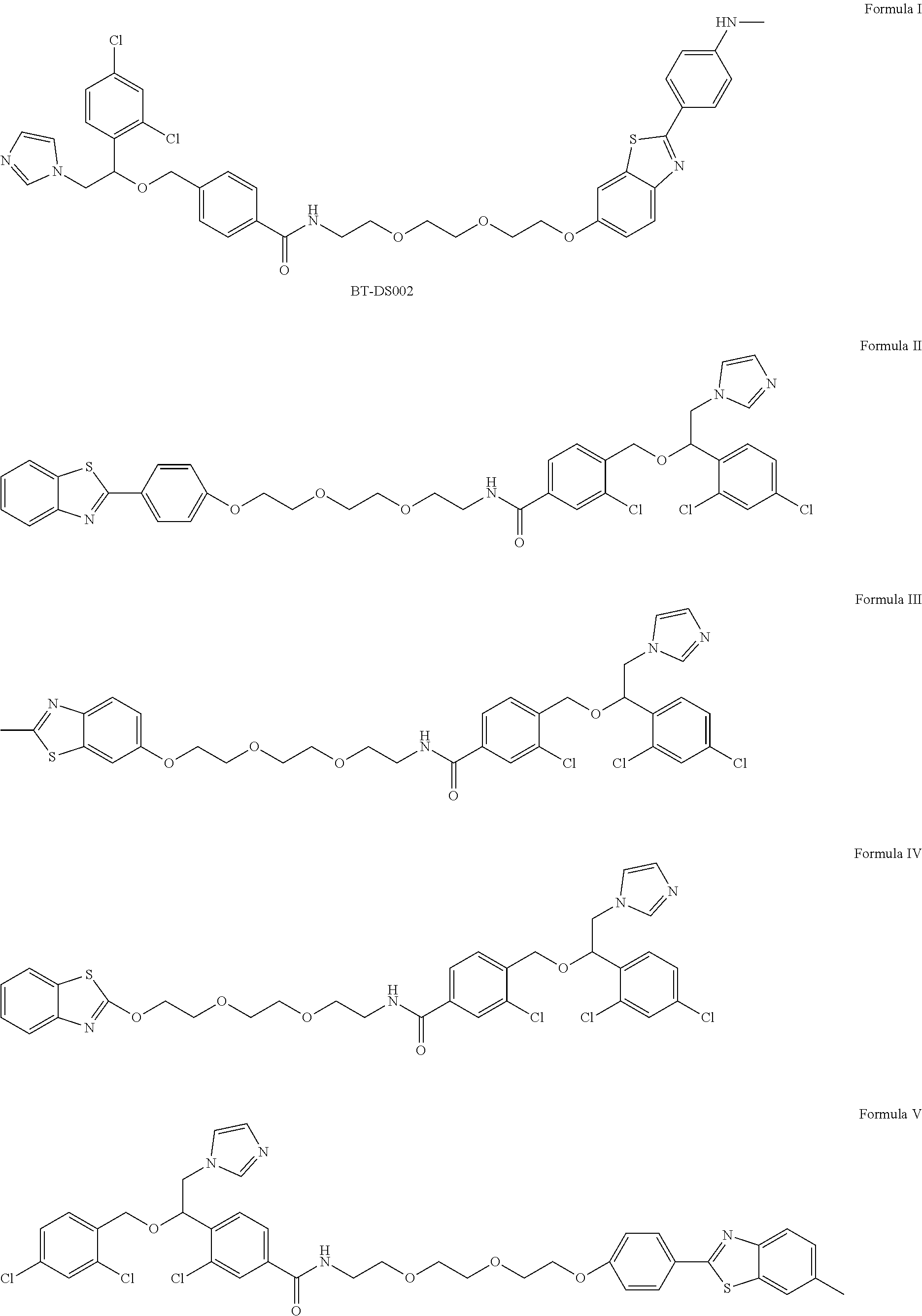

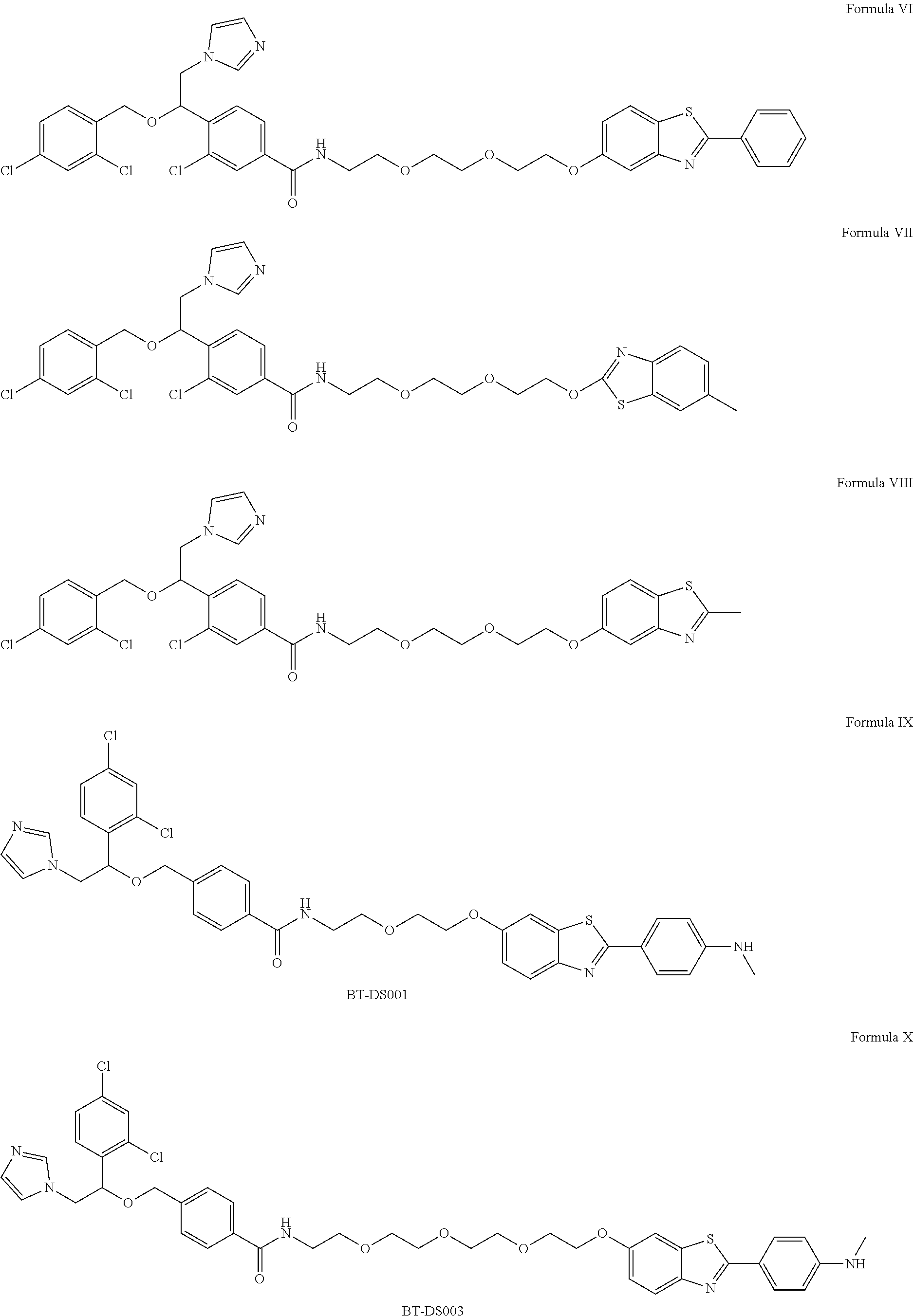

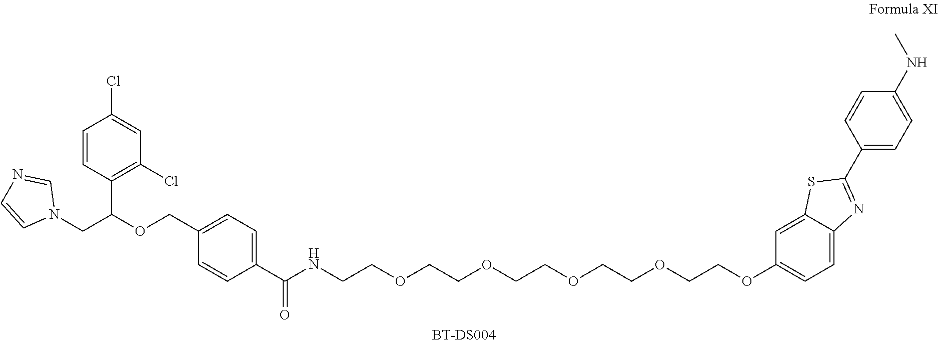

FIGS. 2 to 4 show the results of the functional assay for the compounds of group I, FIG. 2 based on proteasome stimulation in vitro. The biochemical assay was performed with a purified human 20S proteasome. The compounds were incubated with 20S proteasomes for 1 h, and the proteasome activity was measured with a TAS-1 probe. DMSO samples were used as control. FIG. 3 shows the results of the assay for proteasome activity in HEK293 cells for the compounds of group I. The compounds were dosed for 1.5 h, the proteasome activity was measured with Me4BodipyFLAhx3L3VS activity probe, DMSO samples were used as control. FIG. 4 shows the results of the assay for cellular toxicity in HEK293 cells for the compounds of group I. The compounds were dosed for 24 h, the viability measured with CellTiter-Glo, and DMSO was used as control (dashed line). The inventive compounds are better stimulators than the activator alone (MO), especially when they have a longer linker, e.g. (PEG)₄-5. In particular, the degraders DS001 and DS003 show a clear improvement in degrading a-synuclein (compared to DMSO and MO), see FIG. 6. This data involving HEK293 cells suggests that compounds involving different linker length exhibit efficacy.

### II. Tau Degrader and Proteasome Supercharger

The molecules of this group are composed of miconazole as the activator, (PEG)x as the ligand, and Flortaucipir as well as derivatives thereof as the target binder. These compounds are thus designed to promote the degradation of tau with enhanced proteasome activity.

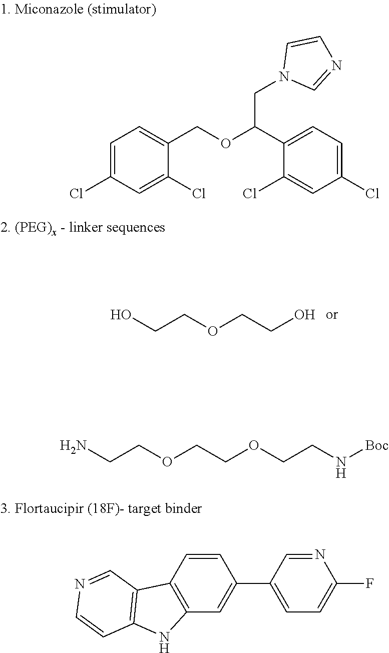 [SMILES: Clc1ccc(COC(Cn2ccnc2)c2ccc(Cl)cc2Cl)c(Cl)c1 ; OCCOCCO ; CC(C)(C)OC(=O)NCCOCCOCCN ; Fc1ccc(-c2ccc3c(c2)[nH]c2ccncc23)cn1]

Flortaucipir 18F (Chun-Fang Xia, et al. [18F]T807, a novel tau positron emission tomography imaging agent for Alzheimer's disease, Alzheimer's & Dementia, Volume 9, Issue 6, 2013, Pages 666-676, ISSN 1552-5260, https://doi.org/10.1016/j.jalz.2012.11.008) is an FDA approved PET agent to monitor tau levels (Eli Lilly product Tauvid). Tauvid binds tau in vivo and preferentially binds to mutated tau forms associated with Alzheimer's disease pathology (David T. Jones, et al. In vivo ¹⁸F-AV-1451 tau PET signal in MAPT mutation carriers varies by expected tau isoforms, Neurology March 2018, 90 (11) e947-e954; DOI: 10.1212/WNL.0000000000005117).

### Group II: Compounds According to the Present Invention

The compounds of group II were generally synthesized as follows:

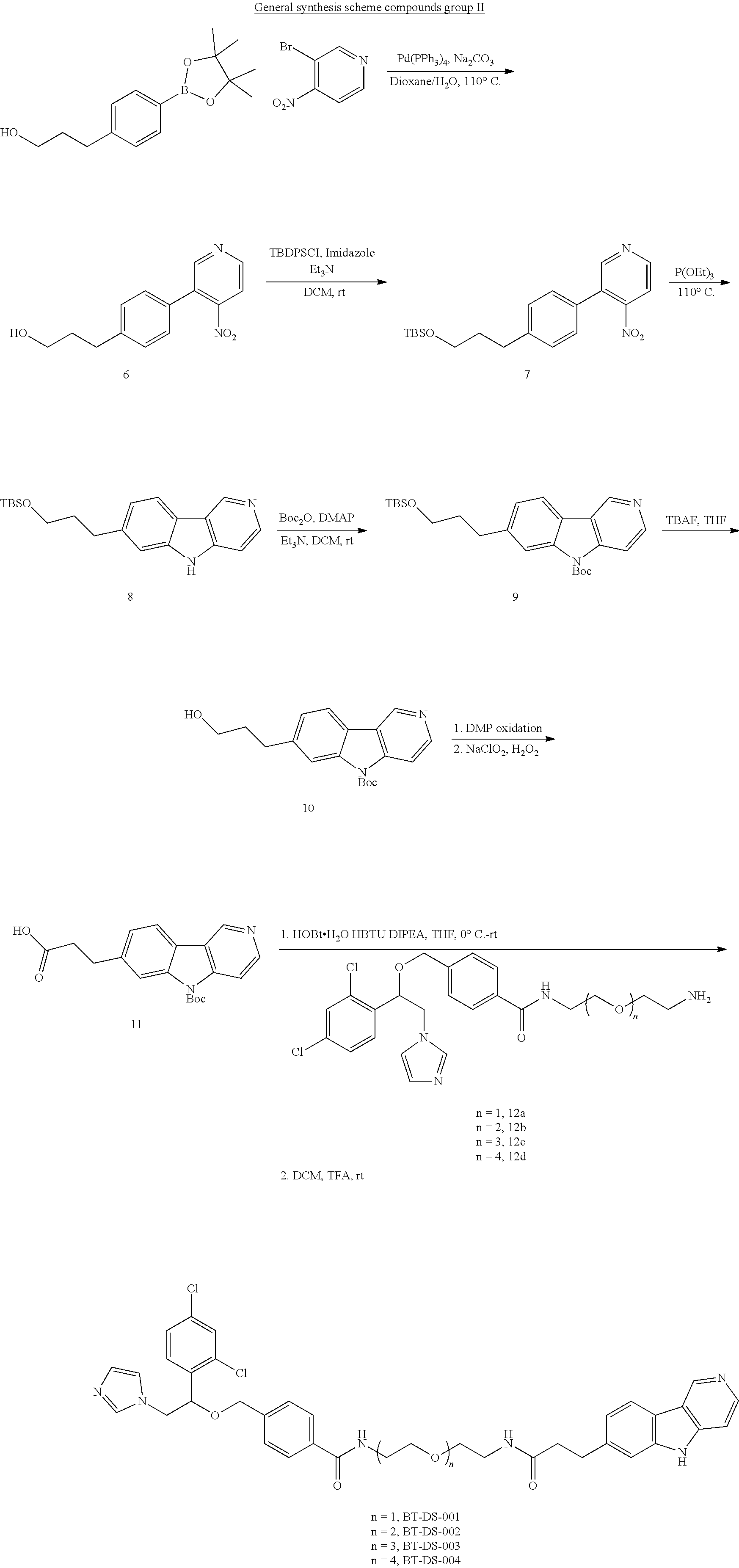

### Compound 6: 3-(4-(4-nitropyridin-3-yl)phenyl)propan-1-ol

A solution of 3-bromo-4-nitropyridine (2 g, 4.93 mmol), 3-(4-(4,4,5,5-tetramethyl-1,3,2-dioxaborolan-2-yl)phenyl)propan-1-ol (2.44 g, 1.1 eq), Na2CO¬3 (2.62 g, 2.5 eq) and Pd(PPh3)4 (569 mg, 0.05 eq) in 1,4-dioxane (40 mL) and H2O (10 mL) was stirred at 110° C. for 16 hr. Thereafter, it was quenched with a saturated solution of NH4Cl and was subsequently extracted with DCM. The combined extracts were dried with anhydrous sodium sulfate and the solvent was removed under reduced pressure to afford the title crude compound as a yellow liquid, which was used for the next step without purification.

### Compound 7: 3-(4-(3-((tert-butyldimethylsilyl)oxy)propyl)phenyl)-4-nitropyridine

In a 250 ml round was added 3-(4-(4-nitropyridin-3-yl)phenyl)propan-1-ol (2.54 g, 1 eq) in DCM (35 mL). Subsequently, imidazole (1.34 g, 2 eq), TBDPSCl (5.4 g, 2 eq) and triethylamine (4.11 ml, 3 eq) were added, and the mixture was stirred at room temperature overnight. After completion, the reaction was quenched with a saturated solution of NH4Cl. The resulting mixture was extracted with DCM and the combined organic phases were washed with brine, dried over sodium sulfate and concentrated under reduced pressure. The residue was purified by flash column chromatography (0-5% MeOH in DCM) to afford the title compound (1.7 g, 34% yield over two steps).

### Compound 8: 7-(3-((tert-butyldimethylsilyl)oxy)propyl)-5H-pyrido[4,3-b]indole

A solution of 3-(4-(3-((tert-butyldimethylsilyl)oxy)propyl)phenyl)-4-nitropyridine (1.7 g, 3.42 mmol) in P(OEt)3 (30 mL) was stirred at 110° C. for 3 hr in a 100 ml 3 neck round bottom flask. After completion was evidenced from TLC, the flask was cooled down to 50° C., and excess P(OEt)3 was distilled out using a short path distillation setup. The residue was purified on silica gel chromatography using 1-5% MeOH in DCM to afford the title compound (700 mg, 44%).

### Compound 9: tert-butyl 7-(3-((tert-butyldimethylsilyl)oxy)propyl)-5H-pyrido[4,3-b]indole-5-carboxylate

To a stirred solution of 7-(3-((tert-butyldimethylsilyl)oxy)propyl)-5H-pyrido[4,3-b]indole (600 mg, 1.29 mmol) and DMAP (97.8 mg, 0.6 eq) in DCM (10 mL) at 25° C. was added Et3N (0.67 ml, 3.7 eq) and (Boc)2O (704.5 mg, 2.5 eq). The reaction mixture was stirred at room temperature overnight and quenched with a solution of saturated NH4Cl. The resulting mixture was extracted with DCM and the combined organic phases were washed with brine, dried over sodium sulfate and concentrated under reduced pressure. The residue was purified by flash column chromatography (0-5% MeOH in DCM) to afford the title compound (463 mg, 64%).

### Compound 10: tert-butyl 7-(3-hydroxypropyl)-5H-pyrido[4,3-b]indole-5-carboxylate

To a stirred solution of tert-butyl 7-(3-((tert-butyldimethylsilyl)oxy)propyl)-5H-pyrido[4,3-b]indole-5-carboxylate (155 mg, 0.274 mmol) in THF (2.5 mL) at 0° C. was added TBAF (1.0 M in THF, 0.411 mL, 1.5 eq) dropwise. The reaction was left to warm up to room temperature and stirred for 2 hrs. The reaction was quenched with acetic acid (0.1 mL). The mixture was concentrated under reduced pressure. The residue was purified by flash column chromatography (0-5% MeOH in DCM, 0.5% triethylamine) to afford the title compound (50 mg, 55%).

### Compound 11: 3-(5-(tert-butoxycarbonyl)-5H-pyrido[4,3-b]indol-7-yl)propanoic acid

To a stirred solution of tert-butyl 7-(3-hydroxypropyl)-5H-pyrido[4,3-b]indole-5-carboxylate (62 mg, 0.19 mmol, 1 eq) in DCM (3 ml) was added NaHCO₃ (32 mg, 2 eq) and Dess-Martin periodinane (161.13 mg, 2 eq), and stirred for 30 min. After completion, water was added to it, and the resulting mixture was extracted with DCM; the combined organic phases were washed with brine, dried over sodium sulfate and concentrated under reduced pressure to afford the crude aldehyde. The above residue was dissolved in THE (1 mL) and t-BuOH (0.5 ml). In a separate vial was added H2O (0.5 ml), NaClO2 (5 eq), NaH2PO4·H2O (5 eq) and H2-02 (1.5 eq). The aqueous solution was then mixed with the aldehyde crude at room temperature. After about 1 hr, the reaction was complete. The mixture was extracted with DCM and the combined organic phases were washed with brine, dried over sodium sulfate and concentrated under reduced pressure. The residue was purified by flash column chromatography (2-5% MeOH in DCM) to afford the title compound (55 mg, 85% over two steps).

### Compound 12a-d: General Procedure for the Synthesis of PEGylated Miconazole Derivatives

In a 20 ml vial was added 4-((1-(2,4-dichlorophenyl)-2-(1H-imidazol-1-yl)ethoxy)methyl)benzoic acid (100 mg, 1 eq), HOBt hydrate (2 eq), EDC hydrochloride (1.2 eq) followed by DCM (5 ml) and DIPEA (4 eq). The contents were stirred for 5 min and then the respective amino Boc-protected linker (1.1 eq) was added to the vial. The reaction mixture was stirred until completion and a saturated solution of NH4C1 was added to the reaction mixture. The aqueous layer was extracted 3 times using DCM; the combined extracts were dried using anhydrous sodium sulfate, filtered and the solvent was removed under reduced pressure. The residue was purified on silica gel using 1-5% MeOH in DCM. The crude was then dissolved in 50% TFA in DCM and stirred for 2 hrs to remove the Boc protecting group. TFA-DCM was removed using a jet of argon, and then DCM was added to the reaction mixture followed by a saturated solution of NaHCO₃ to neutralize TFA. The aqueous layer was extracted 3 times using DCM; the combined extracts were dried using anhydrous sodium sulfate, filtered and the solvent was removed under reduced pressure to afford the title compounds

### Compound BT-DT-(001-004): General Procedure of Amide Coupling

In an 8 ml vial was added 3-(5-(tert-butoxycarbonyl)-5H-pyrido[4,3-b]indol-7-yl)propanoic acid (10 mg, 1 eq), HOBt hydrate (2 eq), HBTU (1.2 eq) followed by DCM (1 ml) and DIPEA (4 eq). The contents were stirred for 30 min (activation step) and then compound 12 (a-d) (1.1 eq) was added to the vial. The reaction mixture was stirred until completion. After completion, a saturated solution of NH4C1 was added to the reaction mixture. The aqueous layer was extracted 3 times using DCM; the combined extracts were dried using anhydrous sodium sulfate, filtered and the solvent was removed under reduced pressure. The crude molecule was dissolved in 50% TFA in DCM and stirred for 2 hrs to remove the Boc protecting group. TFA-DCM was removed using a jet of argon, then DCM was added to the reaction mixture followed by saturated solution of NaHCO₃ to neutralize TFA. The aqueous layer was extracted 3 times using DCM; the combined extracts were dried using anhydrous sodium sulfate, filtered and the solvent was removed under reduced pressure. The compounds were purified on silica using 0-8% MeOH in DCM if need to afford the title compounds

Following the Amide coupling procedure as described above, the following compounds were prepared.

| Exam- | |
|---|---|
| ples | Product/Mass |
| BT- DT001 N-(2- (2- (3-(5H- pyrido [4,3- b] indol- 7-yl) pro- pana- mido) ethoxy) ethyl)- 4-((1- | 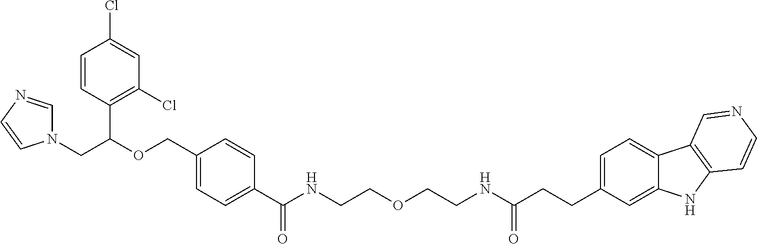 [SMILES: O=C(CCc1ccc2c(c1)[nH]c1ccncc12)NCCOCCNC(=O)c1ccc(COC(Cn2ccnc2)c2ccc(Cl)cc2Cl)cc1] |
| (2,4- | |
| di- | 699.2 (M + H+) |
| chloro- | 350.2 (M + 2H+)/2 |
| phenyl)- | |
| 2-(1H- | |
| imida- | |
| zol-1- | |
| yl) | |
| ethoxy) | |
| methyl) | |
| benza- | |
| mide | |
| BT- DT002 N-(2- (2- (2-(3- (5H- pyrido [4,3-b] indol- 7-yl) pro- pana- mido) ethoxy) ethoxy) ethyl)- 4-((1- (2,4- | 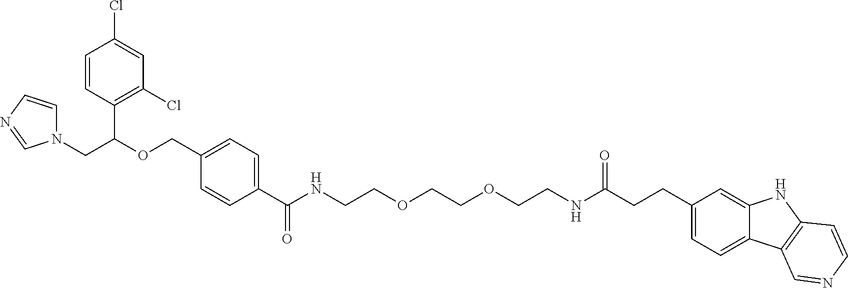 [SMILES: O=C(CCc1ccc2c(c1)[nH]c1ccncc12)NCCOCCOCCNC(=O)c1ccc(COC(Cn2ccnc2)c2ccc(Cl)cc2Cl)cc1] |
| di- | |
| chloro- | 743.0 (M + H+) |
| phenyl)- | 372.2 (M + 2H+)/2 |
| 2-(1H- | |
| imida- | |
| zol-1- | |
| yl) | |
| ethoxy) | |
| methyl) | |
| benza- | |
| mide | |
| BT- DT003 4-((1- (2,4- di- chloro- phenyl)- 2-(1H- imida- zol-1- yl) ethoxy) methyl)- | 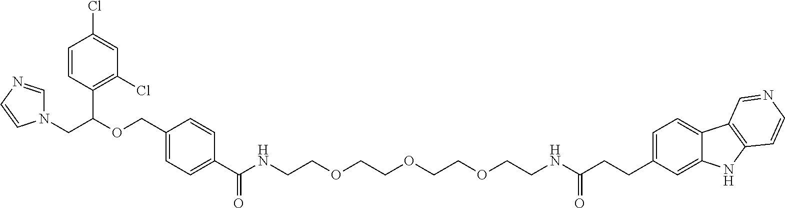 [SMILES: O=C(CCc1ccc2c(c1)[nH]c1ccncc12)NCCOCCOCCOCCNC(=O)c1ccc(COC(Cn2ccnc2)c2ccc(Cl)cc2Cl)cc1] |
| N-(13- | |
| oxo- | 787.3 (M + H+) |
| 15-(5H- | |
| pyrido | |
| [4,3- | |
| b]indol- | |
| 7-yl)- | |
| 3,6,9- | |
| trioxa- | |
| 12- | |
| aza- | |
| penta- | |
| decyl) | |
| benza- | |
| mide | |
| BT- | 831.3 (M + H+) |
| DT004 | 416.2 (M + 2H+)/2 |
| 4-((1- | |
| (2,4- | |
| di- | |
| chloro- | |
| phenyl)- | |
| 2-(1H- | |
| imida- | |
| zol-1- | |
| yl) | |
| ethoxy) | |
| methyl)- | |
| N-(16- | |
| oxo-18- | |
| (5H- | |
| pyrido | |
| [4,3- | |
| b]indol- | |
| 7-yl)- | |
| 3,6,9,12- | |
| tetraoxa- | |
| 15- | |
| azaocta- | |
| decyl) | |
| benza- | |
| mide | |

### Compounds of Group II

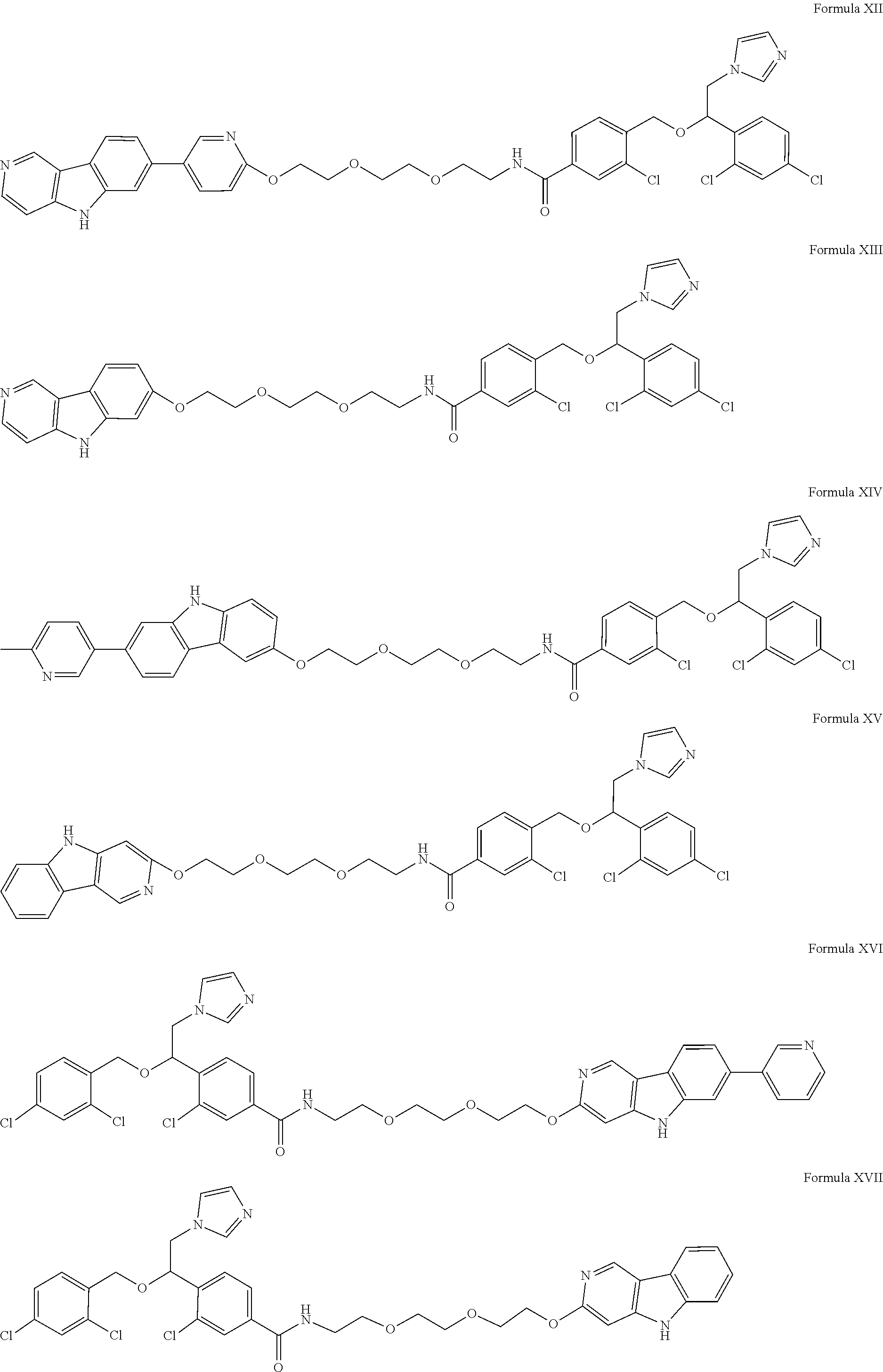

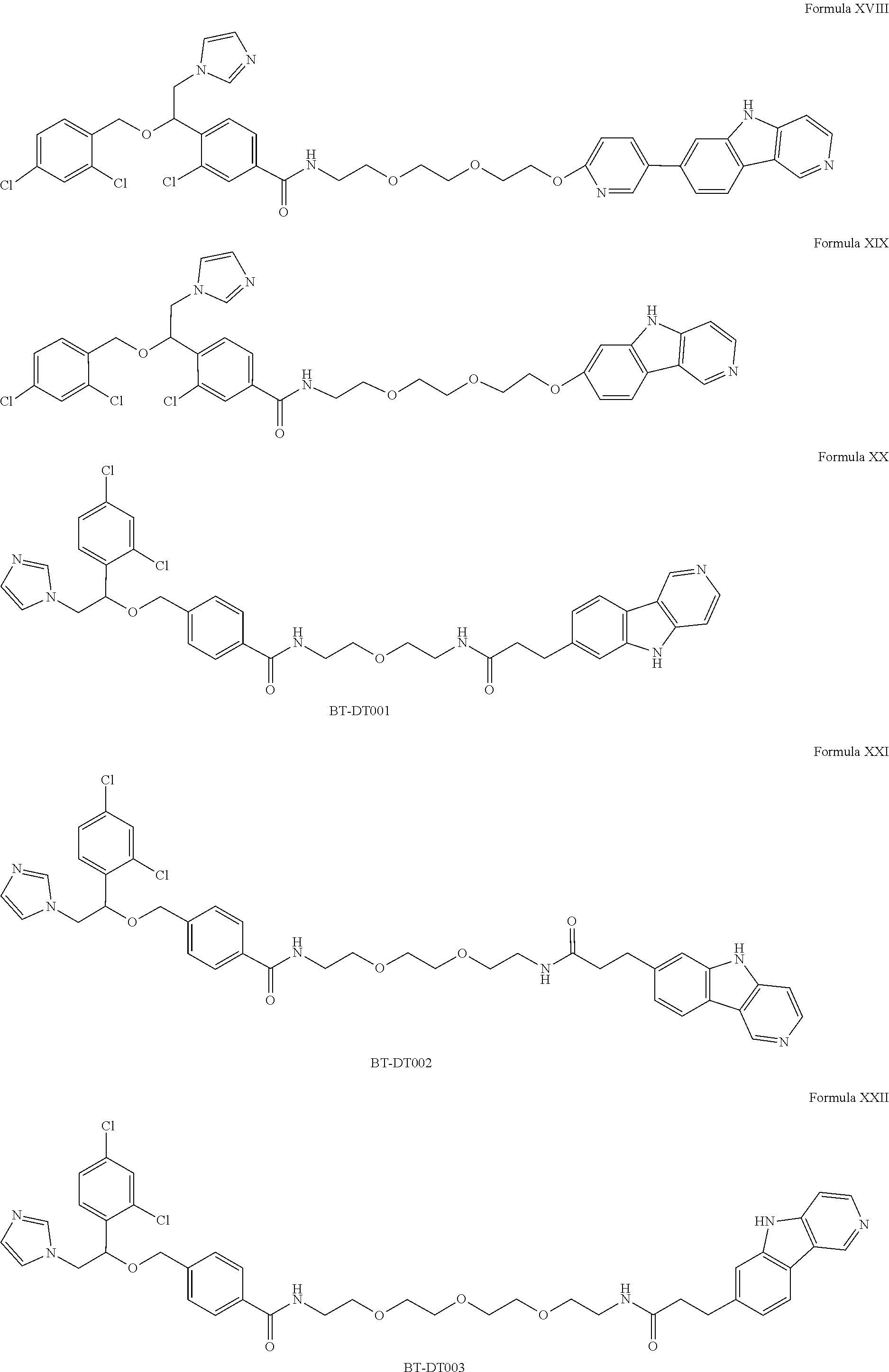

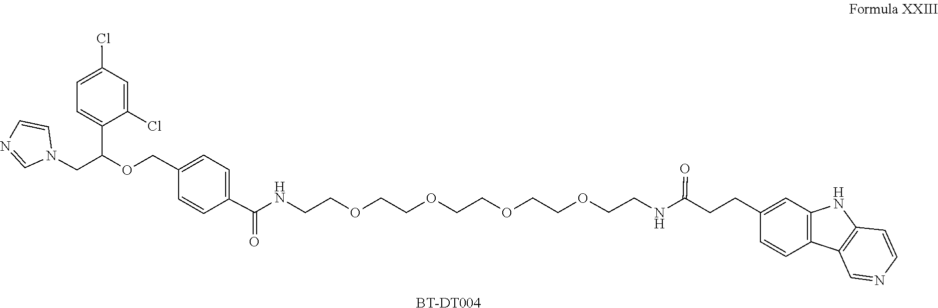 [SMILES: O=C(CCc1ccc2c(c1)[nH]c1ccncc12)NCCOCCOCCOCCOCCNC(=O)c1ccc(COC(Cn2ccnc2)c2ccc(Cl)cc2Cl)cc1 ; "BT-DT004"]

FIG. 5 shows the results of the functional assay for the compounds of group II, based on proteasome stimulation in vitro. The biochemical assay was performed with a purified human 20S proteasome. The compounds were incubated with 20S proteasomes for 1 h, and the proteasome activity was measured with a TAS-1 probe. DMSO samples were used as control (dashed line). The inventive compounds are better stimulators than the activator alone (MO), especially when they have a longer linker, e.g., (PEG)₄₋₅. FIG. 7 shows the results of the functional assay for the compounds of group II, proteasome stimulation in cells. For proteasome activity in HEK293 cells, the compounds were dosed for 1.5 h, and the proteasome activity measured with Me4BodipyFLAhx3L3VS activity probe. DMSO samples were used as control (dashed line). FIG. 8 shows the results of the cell toxicity assay in HEK293 cells for the compounds of group II. Compounds were dosed for 24 h, and the viability measured with CellTiter-Glo. DMSO samples were used as control (dashed line). FIG. 9 shows the results of the functional assay for the compounds of group II, tau degradation in HEK293 cells. After tau transfection for 48 h, the compounds were dosed for 16 h at 25 μM together with 30 μM cycloheximide, and total tau was determined (western blot). DMSO samples were used as control (dashed line). Preferred compound DT-001 shows clear tau degradation improvement. This data involving HEK293 cells suggests that compounds involving different linker length exhibit efficacy.

### III. Amyloid Degrader and Proteasome Supercharger

The molecules of this group are composed of miconazole as the activator, (PEG)x as the ligand, and Florbetapir as well as derivatives thereof as the target binder. These compounds are thus designed to promote the degradation of beta-amyloids with enhanced proteasome activity.

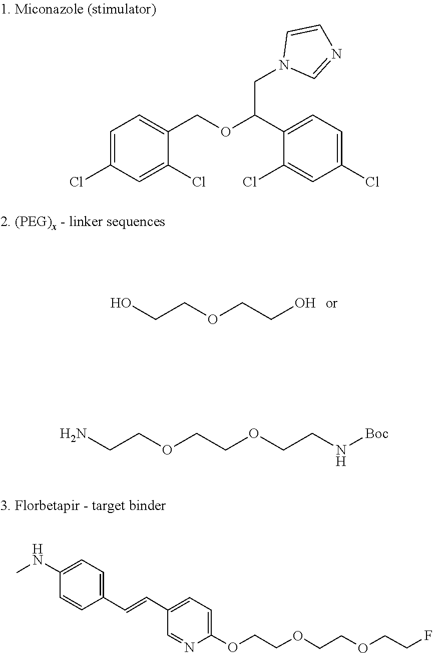 [SMILES: Clc1ccc(COC(Cn2ccnc2)c2ccc(Cl)cc2Cl)c(Cl)c1 ; OCCOCCO ; CC(C)(C)OC(=O)NCCOCCOCCN ; CNc1ccc(/C=C/c2ccc(OCCOCCOCCF)nc2)cc1]

Florbetapir F18 (Wei Zhang, et al. 18F-labeled styrylpyridines as PET agents for amyloid plaque imaging, Nuclear Medicine and Biology, Volume 34, Issue 1, 2007, Pages 89-97, ISSN 0969-8051, https://doi.org/10.1016/j.nucmedbio.2006.10.003) is an FDA approved PET agent to monitor beta-amyloid levels (Eli Lilly product—Amyvid). Amyvid binds beta-amyloid in vivo (John Lister-James, et al. Florbetapir F-18: A Histopathologically Validated Beta-Amyloid Positron Emission Tomography Imaging Agent, Seminars in Nuclear Medicine, Volume 41, Issue 4, 2011, Pages 300-304, ISSN 0001-2998, https://doi.org/10.1053/j.semnuclmed.2011.03.001).

The compounds of Group III were generally synthesized as follows.

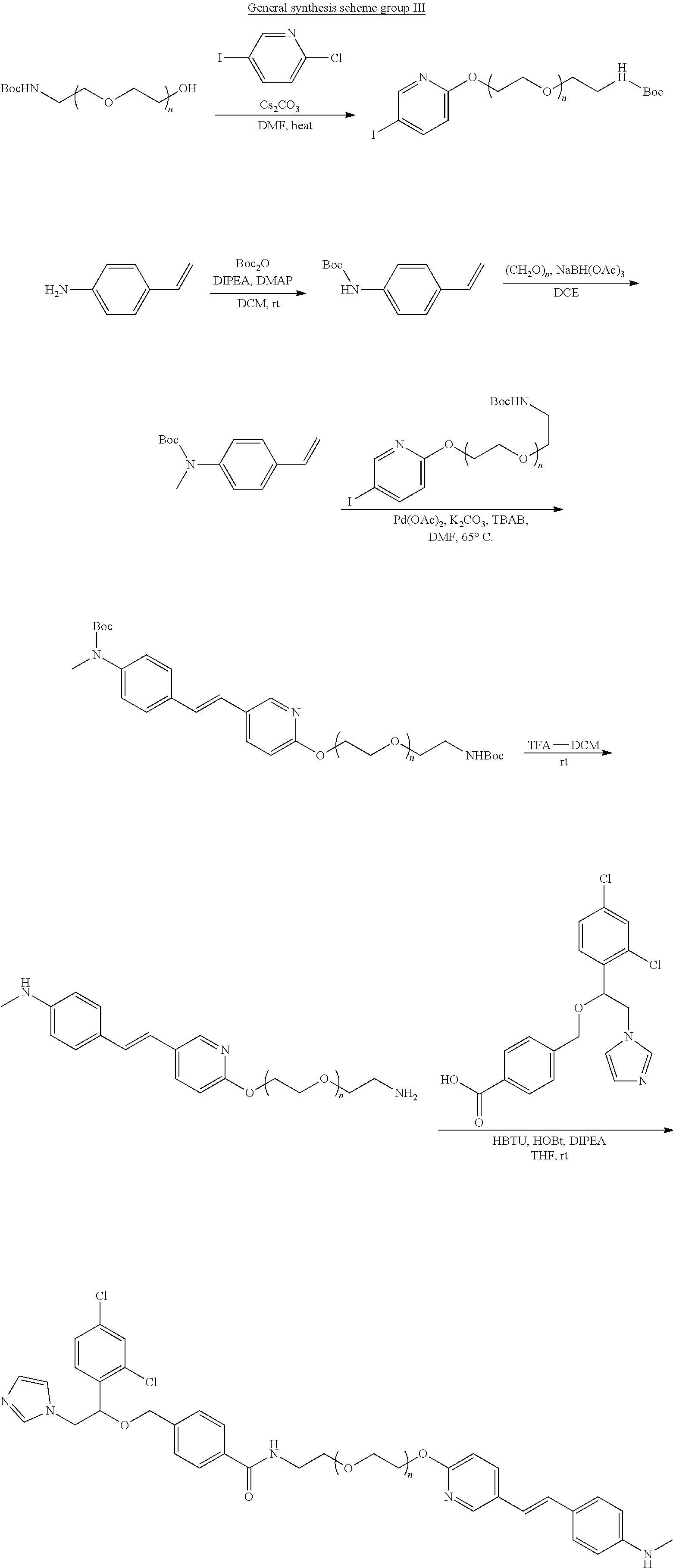

### Group III: Compounds According to the Present Invention

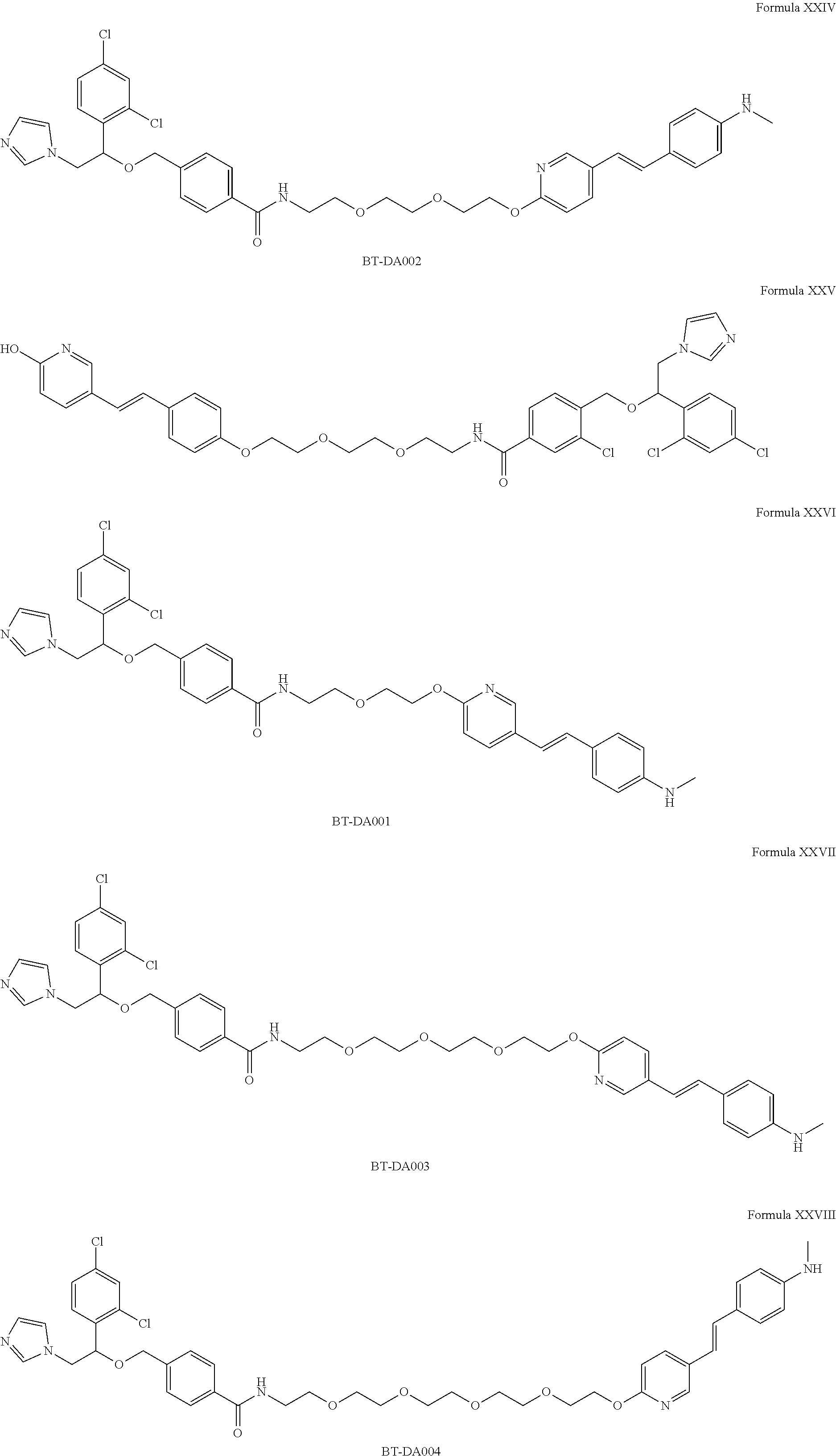

A biochemical assay can be performed for these compounds with a purified human 20S proteasome as described herein previously. The compounds may be incubated with 20S proteasomes for 1 h, and the proteasome activity is measured with e.g. a TAS-1 probe. DMSO samples can be used as control. The inventive compounds are expected to be better stimulators than the activator alone (MO). For measuring proteasome activity in HEK293 cells, the compounds are dosed for 1.5 h, and the proteasome activity can be measured with e.g. Me4BodipyFLAhx3L3VS activity probe. DMSO samples can be used as control. Cell toxicity assay can be performed in HEK293 cells for the compounds of group III. Compounds are e.g. dosed for 24 h, and the viability is e.g. measured with CellTiter-Glo. DMSO samples can be used as control. As functional assay for the compounds of group II, beta-amyloid degradation in HEK293 cells can be measured. After beta-amyloid transfection for e.g. 48 h, the compounds may be dosed for 16 h at 25 μM together with 30 μM cycloheximide, and total beta-amyloid can be determined. DMSO samples can be used as control. The compounds are expected to show clear beta-amyloid degradation improvement.

The inventive compounds are better stimulators than the activator (S) alone, especially when they have a longer linker, i.e. (PEG)₅.

1. A molecule improving the degradation of a proteinaceous target molecule by the proteasome of a cell, according to the general formula
B-L-S
wherein
B indicates at least one binding moiety binding or promoting binding to the target molecule,
S indicates at least one moiety stimulating proteasome degradation, and
L indicates at least one linker moiety suitably linking said at least one binding moiety with said at least one moiety stimulating proteasome degradation, and
pharmaceutically acceptable salts thereof,
wherein the at least one moiety stimulating proteasome degradation (S) is selected from the group consisting of a compound stimulating degradation in an ubiquitin-independent manner, a compound stimulating the 20S core particle (CP) of the proteasome, miconazole, and derivatives thereof.

2. The molecule according to claim 1, wherein the target molecule is selected from the group consisting of an accumulated undesired protein, an accumulated pathological protein, prions, beta-amyloid, tau, alpha-synuclein, estrogen receptor alpha (ERα), androgen receptor (AR), huntingtin, ataxin, methionine aminopeptidase-1 (MetAP-2), cellular retinoic acid-binding protein-I, cellular retinoic acid-binding protein-II (CRABP-II), alpha 1 antitrypsin, rhodopsin, crystallin, transthyretin, amyloid, cystic fibrosis transmembrane conductance regulator (CTFR), superoxide dismutase 1 (SOD1), transactive response DNA binding protein 43 kDa (TDP-43), fused in sarcoma (FUS), and islet amyloid polypeptide (IAPP).

3. The molecule according to claim 1, wherein the at least one binding moiety (B) binding or promoting binding to the target molecule is selected from the group consisting of an antibody that is specific for said target molecule or a specific binding fragment thereof, a proteinaceous group specifically binding to said target molecule, a binding peptide, a natural or non-natural nucleic acid, a small molecule specifically binding to said target molecule, florbetapir F18, amyvid, tauvid, and thioflavin.

4. The molecule according to claim 1, wherein the at least one linker moiety (L) suitably linking said at least one binding moiety with said at least one moiety stimulating proteasome degradation is selected from the group consisting of a branched or unbranched chemical linker compound, a branched or unbranched linker amino acid sequence, a cleavable branched or unbranched linker compound, and a labelled branched or unbranched linker compound.

5. The molecule according to claim 1, selected from the group of the compounds according to any one of formulae I to XXVIII, and pharmaceutically acceptable salts and derivatives thereof.

6. The molecule according to claim 1, wherein said molecule is bispecific.

7. The molecule according to claim 1, wherein said molecule synergistically improves the degradation of the proteinaceous target molecule.

8. The molecule according to claim 1, additionally comprising at least one moiety binding to the proteasome 19S subunit.

9. A method for producing a molecule according to claim 1, comprising suitably linking the at least one binding moiety (B) to the at least one moiety stimulating proteasome degradation (S) through the at least one linker moiety (L).

10. The method according to claim 9, further comprising the step of identifying the at least one binding moiety (B) and/or the at least one moiety stimulating proteasome degradation (S) before the linking thereof to the at least one linker moiety (L).

11. A pharmaceutical composition, comprising the molecule according to claim 1, or a pharmaceutically acceptable salt thereof, together with at least one pharmaceutically acceptable carrier or buffer.

12-13. (canceled)

14. A method for improving proteasome function wherein said method comprises contacting a proteasome with a molecule according to claim 1.

15. A method for removing an undesired proteinaceous target wherein said method comprises the use of a molecule according to claim 1.

16. A method for treating or preventing a disease or condition in a mammalian subject, wherein said method comprises administering, to a subject in need of said treatment or prevention, an effective amount of the molecule according to claim 1.

17. The method according to claim 16, wherein the disease or condition to be treated or prevented is selected from the group consisting of a disease or condition caused by an undesired proteinaceous target molecule, caused by an accumulated pathological protein, proteopathies, alpha-synucleinopathies, tauopathies, neurodegenerative diseases associated with alpha-synuclein accumulation or aggregation, neurodegenerative diseases associated with tau accumulation or aggregation, neurodegenerative diseases associated with beta-amyloid accumulation or aggregation, Parkinson's disease, Alzheimer's disease, dementia, dementia with Lewy bodies, frontotemporal dementia, progressive supranuclear palsy, Pick's disease, amyotrophic lateral sclerosis, Huntington's disease, spinocerebellar ataxias, prion diseases, cystic fibrosis, alpha 1 antitrypsin deficiency, diabetes type-2, retinitis pigmentosa, cataracts, amyloidosis, desmin-related cardiomyopathy, multiple system atrophy, cancer, breast cancer, prostate cancer, cancer tumor metastasis, and ageing.

18. The method according to claim 16, wherein the subject is a human.

19. The molecule according to claim 4, wherein the linker is a polyethylene linker sequence.
